(19) United States
(12) Patent Application Publication
(10) Pub. No.: US 2024/0360052 A1
(43) Pub. Date: Oct. 31, 2024

(54) PHOSPHINE-IMINO-QUINOLINE AND RELATED LIGANDS FOR USE IN ETHYLENE OLIGOMERIZATION PROCESSES

(71) Applicant: Chevron Phillips Chemical Company LP, The Woodlands, TX (US)

(72) Inventors: Michael Webster-Gardiner, Humble, TX (US); Brooke L. Small, Kingwood, TX (US); Julie A. Leseberg, Kingwood, TX (US)

(21) Appl. No.: 18/642,892

(22) Filed: Apr. 23, 2024

Related U.S. Application Data
(60) Provisional application No. 63/498,538, filed on Apr. 27, 2023.

(51) Int. Cl. C07C 2/24; B01J 23/745; B01J 23/75; C07C 11/02; C07D 215/36; C07F 9/576

(52) U.S. Cl. CPC C07C 2/24; B01J 23/745; B01J 23/75; C07C 11/02; C07D 215/36; C07F 9/5765; C07C 2521/04; C07C 2523/745; C07C 2523/75

(57) ABSTRACT

Catalyst compositions containing phosphine-imino-quinoline ligands and transition metal complexes having phosphine-imino-quinoline ligands are disclosed. These catalyst compositions can be used in ethylene oligomerization processes to produce 1-hexene and 1-octene.

### REFERENCE TO RELATED APPLICATION

This application claims the benefit of U.S. Provisional Patent Application No. 63/498,538, filed on Apr. 27, 2023, the disclosure of which is incorporated herein by reference in its entirety.

### FIELD OF THE INVENTION

The present disclosure relates generally to heteroatomic ligands and heteroatomic ligand transition metal compound complexes and their use in catalyst compositions and ethylene oligomerization processes.

### BACKGROUND OF THE INVENTION

Alpha olefins such as 1-hexene and 1-octene can be produced using an ethylene reactant and various combinations of catalyst systems and oligomerization processes. It can be beneficial for the catalyst system employed to have high catalytic activity and thermal stability, as well as being more selective to desirable C₆-C₈ linear α-olefins. Accordingly, it is to these ends that the present invention is generally directed.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Disclosed herein are catalyst compositions and methods for using the catalyst compositions to oligomerize olefins. In particular, the present invention relates to catalyst compositions and to ethylene oligomerization processes that utilize heteroatomic ligands and heteroatomic ligand transition metal compound complexes. In one aspect of this invention, the ligands and complexes can have formulas (IA) and (IB):

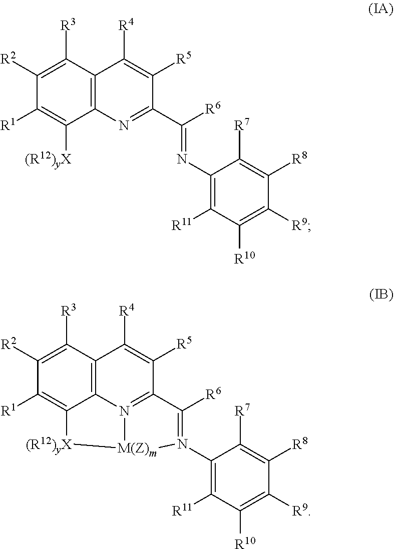

In formulas (IA) and (IB), X can be P or S; y can be equal to 1 when X is S and y can be equal to 2 when X is P; R¹ to R¹¹ independently can be H, a halogen, a nitro group, a C₁-C₁₈ hydrocarbyl group, or a C₁-C₁₈ halogenated hydrocarbyl group, and R⁵ and R⁶ can be joined to form a ring or ring system; and each R¹² independently can be a C₁-C₁₈ hydrocarbyl group or a C₁-C₁₈ halogenated hydrocarbyl group. Referring to formula (IB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group.

In another aspect of this invention, the heteroatomic ligands and heteroatomic ligand transition metal compound complexes can have formulas (IIA) and (IIB):

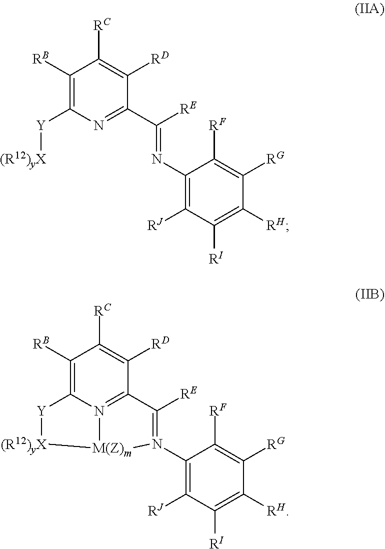

In formulas (IIA) and (IIB), X can be P or S; y can be equal to 1 when X is S and y can be equal to 2 when X is P; Y can be O, NH, or CH₂; R^{B} to R^{J} independently can be H, a halogen, a nitro group, a C₁-C₁₈ hydrocarbyl group, or a C₁-C₁₈ halogenated hydrocarbyl group, and R^{D} and R^{E} can be joined to form a ring or ring system; and each R^{A} independently can be a C₁-C₁₈ hydrocarbyl group or a C₁-C₁₈ halogenated hydrocarbyl group. Referring to formula (IIB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group.

Both the foregoing summary and the following detailed description provide examples and are explanatory only. Accordingly, the foregoing summary and the following detailed description should not be considered to be restrictive. Further, features or variations can be provided in addition to those set forth herein. For example, certain aspects can be directed to various feature combinations and sub-combinations described in the detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description.

FIG. 1 presents a ¹H NMR plot of Ligand 9.

FIG. 2 presents a ¹H NMR plot of Ligand 10.

FIG. 3 presents a ¹H NMR plot of Ligand 11.

FIG. 4 presents a ¹H NMR plot of Ligand 12.

FIG. 5 presents a ¹H NMR plot of Ligand 13.

FIG. 6 presents a ¹H NMR plot of Ligand 14.

FIG. 7 presents a ¹H NMR plot of Ligand 15.

FIG. 8 presents a ¹H NMR plot of Ligand 16.

FIG. 9 presents a ¹H NMR plot of Ligand 17.

FIG. 10 presents a ¹H NMR plot of Ligand 18.

FIG. 11 presents a ¹H NMR plot of Ligand 19.

FIG. 12 presents a ¹H NMR plot of Ligand 20.

FIG. 13 presents a ¹H NMR plot of Ligand 21.

FIG. 14 presents a ¹H NMR plot of Ligand 22.

FIG. 15 presents a ¹H NMR plot of Ligand 23, and FIG. 16 presents a zoomed-in view of the high chemical shift region of FIG. 15.

FIG. 17 presents a ¹H NMR plot of Ligand 24, and FIG. 18 presents a zoomed-in view of the high chemical shift region of FIG. 17.

FIG. 19 presents a ¹H NMR plot of Ligand 25, and FIG. 20 presents a zoomed-in view of the high chemical shift region of FIG. 19.

While the inventions disclosed herein are susceptible to various modifications and alternative forms, only a few specific aspects have been shown by way of example in the drawings and described in detail below. The figures and detailed descriptions of these specific aspects are not intended to limit the breadth or scope of the inventive concepts or the appended claims in any manner. Rather, the figures and detailed descriptions are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present invention can comprise; alternatively, can consist essentially of; or alternatively, can consist of; a heteroatomic ligand, a transition metal compound, and an organoaluminum compound. The terms “a,” “an,” and “the” are intended to include plural alternatives, e.g., at least one, unless otherwise specified. For instance, the disclosure of “an organoaluminum compound” is meant to encompass one, or mixtures or combinations of two or more, organoaluminum compound(s), unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastercomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “oligomer” refers to a compound that contains from 2 to 20 monomer units. The terms “oligomerization product” and “oligomer product” include all products made by the “oligomerization” process, including the “oligomers” and products which are not “oligomers” (e.g., products which contain more than 20 monomer units, or solid polymer), but exclude other non-oligomer components of an oligomerization reactor effluent stream, such as unreacted ethylene, organic reaction medium, and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the active catalytic site, or the fate of the organoaluminum compound and the heteroatomic ligand transition metal compound complex (or the organoaluminum compound and the heteroatomic ligand and the transition metal compound) after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, when a chemical moiety having a certain number of carbon atoms is disclosed or claimed, the intent is to disclose or claim individually every possible number that such a range could encompass, consistent with the disclosure herein. For example, the disclosure that a moiety is a C₁ to C₁₈ hydrocarbyl group, or in alternative language, a hydrocarbyl group having from 1 to 18 carbon atoms, as used herein, refers to a moiety that can have 1, 2, 3, 4, 5, 6, 7, 8, 9, 10, 11, 12, 13, 14, 15, 16, 17, or 18 carbon atoms, as well as any range between these two numbers (for example, a C₁ to C₈ hydrocarbyl group), and also including any combination of ranges between these two numbers (for example, a C₂ to C₄ and a C₁₂ to C₁₆ hydrocarbyl group).

Similarly, another representative example follows for the molar ratio of Al:transition metal in the catalyst composition. By a disclosure that the molar ratio can range from 10:1 to 5,000:1, the intent is to recite that the molar ratio can be any ratio within the range and, for example, can include any range or combination of ranges from 10:1 to 5,000:1, such as from 50:1 to 3,000:1, from 50:1 to 3,000:1, from 75:1 to 2,000:1, from 100:1 to 2,000:1, or from 100:1 to 1,000:1, and so forth. Likewise, all other ranges disclosed herein should be interpreted in a manner similar to these examples.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Disclosed herein are heteroatomic ligand transition metal compound complexes as well as heteroatomic ligands and transition metal compounds, catalyst compositions containing the respective complexes (or the respective ligands and compounds), and ethylene oligomerization processes utilizing the catalyst compositions to produce 1-hexene and/or 1-octene.

An objective of this invention is to develop catalyst systems using heteroatomic ligand transition metal compound complexes (or heteroatomic ligands and transition metal compounds) that provide excellent catalyst activity in ethylene oligomerization processes, resulting in high yields of oligomer products. Another objective of this invention is to develop catalyst systems using heteroatomic ligand transition metal compound complexes (or heteroatomic ligands and transition metal compounds) that are selective in the formation of desirable C₆-C₈ linear α-olefins in ethylene oligomerization processes.

### Ligands and Complexes

In one aspect of this invention, heteroatomic ligands and heteroatomic ligand transition metal compound complexes encompassed herein can have formulas (IA) and (IB):

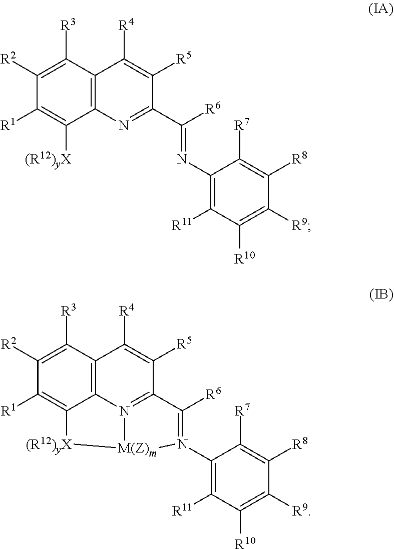

In another aspect of this invention, heteroatomic ligands and heteroatomic ligand transition metal compound complexes encompassed herein can have formulas (IIA) and (IIB):

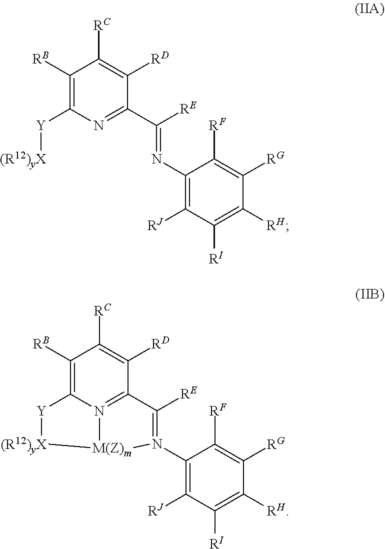

Within formula (IA), X, y, R¹ to R¹¹, and each R¹² are independent elements of the heteroatomic ligand compound. Accordingly, the heteroatomic ligand compound having formula (IA) can be described using any combination of X, y, R¹ to R¹¹, and R¹² disclosed herein. Similarly, within formula (IB), X, y, M, m, each Z, R¹ to R¹¹, and each R¹² are independent elements of the heteroatomic ligand transition metal compound complex. Accordingly, the heteroatomic ligand transition metal compound complex having formula (IB) can be described using any combination of X, y, M, m, Z, R¹ to R¹¹, and R¹² disclosed herein.

Likewise, within formula (IIA), X, y, Y, R^{B} to R^{J}, and each R^{A} are independent elements of the heteroatomic ligand compound. Accordingly, the heteroatomic ligand compound having formula (IIA) can be described using any combination of X, y, Y, R^{B} to R^{J}, and R^{A} disclosed herein. Similarly, within formula (IIB), X, y, Y, M, m, each Z, R^{B} to R^{J}, and each R^{A} are independent elements of the heteroatomic ligand transition metal compound complex. Accordingly, the heteroatomic ligand transition metal compound complex having formula (IIB) can be described using any combination of X, y, Y, M, m, Z, R^{B} to R^{J}, and R^{A} disclosed herein.

Unless otherwise specified, formulas (IA), (IB), (IIA), and (IIB) above, any other structural formulas disclosed herein, and any ligand, complex, compound, or species disclosed herein are not designed to show stereochemistry or isomeric positioning of the different moieties (e.g., these formulas are not intended to display rac or meso isomers, or R or S diastereoisomers), although such compounds are contemplated and encompassed by these formulas and/or structures, unless stated otherwise.

Referring first to formulas (IA) and (IB), X can be P or S; y can be equal to 1 when X is S and y can be equal to 2 when X is P; R¹ to R¹¹ independently can be H, a halogen, a C₁-C₁₈ hydrocarbyl group, or a C₁-C₁₈ halogenated hydrocarbyl group, and R⁵ and R⁶ can be joined to form a ring or ring system; and each R¹² independently can be a C₁-C₁₈ hydrocarbyl group or a C₁-C₁₈ halogenated hydrocarbyl group. Referring to formula (IB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group.

In one aspect, X can be P and y can equal 2 in formulas (IA) and (IB), while in another aspect, X can be S and y can equal 1 in formulas (IA) and (IB). R¹ to R¹¹ independently can be H, a halogen, a C₁-C₁₈ hydrocarbyl group, or a C₁-C₁₈ halogenated hydrocarbyl group, and R⁵ and R⁶ can be joined to form a ring or ring system in formulas (IA) and (IB). It is contemplated that any of R¹ to R¹¹ can be either the same or different.

For example, R¹ to R¹¹ independently can be H, a halogen (e.g., F, Cl, Br), a C₁-C₁₈ hydrocarbyl group, or a C₁-C₁₈ halogenated hydrocarbyl group, and the hydrocarbyl and halogenated hydrocarbyl groups, independently, can be C₁-C₁₂ groups, or C₁-C₁₀ groups, C₁-C₈ groups, C₁-C₆ groups, or C₁-C₄ groups. Any hydrocarbyl group, independently, can be an alkyl group, a cycloalkyl group, an aryl group (e.g., a phenyl group or a naphthyl group, optionally substituted), or an aralkyl group (e.g., a benzyl group, optionally substituted), and likewise for halogenated hydrocarbyl groups.

In an aspect, R¹ to R¹¹ independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). For instance, R¹ to R⁶ independently can be H or a methyl group. Additionally or alternatively, R⁷ to R¹¹ independently can be, in some aspects, H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group (—NO₂), a pentafluorophenyl group, or a trifluoromethyl group (CF₃). Additionally or alternatively, R⁵ and R⁶ can be joined to form a ring or ring system. Likewise, in some aspects, R⁷ and R⁸ can be joined to form a ring or ring system.

Each R¹² in formulas (IA) and (IB) independently can be a C₁-C₁₈ hydrocarbyl group or a C₁-C₁₈ halogenated hydrocarbyl group, and any hydrocarbyl and halogenated hydrocarbyl options noted herein for R¹ to R¹¹ also can apply to R¹². Accordingly, in one aspect, each R¹² independently can be a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group (one or more methyl substituents), a naphthyl group, a benzyl group, a pentafluorophenyl group, or a trifluoromethyl group (CF₃). In another aspect, each R¹² independently can be a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

Referring now to the complex of formula (IB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group. The metal in formula (IB), M, can be Fe, Co, or Cr, and thus in one aspect, for instance, M can be Fe or Cr, while in another aspect, M can be Fe; alternatively, M can be Co; or alternatively, M can be Cr.

Each Z in formula (IB) independently can be any suitable monoanionic ligand, such as H, a halogen, a C₁-C₁₈ hydrocarbyl group, or a C₁-C₁₈ halogenated hydrocarbyl group, and any halogen, hydrocarbyl, and halogenated hydrocarbyl options noted herein for R¹ to R¹¹ also can apply to Z. Accordingly, in one aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). In another aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a pentafluorophenyl group, or a trifluoromethyl group (CF₃). In yet another aspect, each Z can be Cl. In still another aspect, two or more Z can be joined to form a dianionic or trianionic or polyanionic ligand (e.g., catechol or a chelating group) to balance the oxidation state of M.

Referring now to formulas (IIA) and (IIB), X can be P or S; y can be equal to 1 when X is S and y can be equal to 2 when X is P; Y can be O, NH, or CH₂; R^{B} to R^{J} independently can be H, a halogen, a C₁-C₁₈ hydrocarbyl group, or a C₁-C₁₈ halogenated hydrocarbyl group, and R^{D} and R^{E} can be joined to form a ring or ring system; and each R^{A} independently can be a C₁-C₁₈ hydrocarbyl group or a C₁-C₁₈ halogenated hydrocarbyl group. Referring to formula (IIB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group.

In one aspect, X can be P and y can equal 2 in formulas (IIA) and (IIB), while in another aspect, X can be S and y can equal 1 in formulas (IIA) and (IIB). R^{B} to R^{J} independently can be H, a halogen, a C₁-C₁₈ hydrocarbyl group, or a C₁-C₁₈ halogenated hydrocarbyl group, and R^{D} and R^{E} can be joined to form a ring or ring system in formulas (IIA) and (IIB). It is contemplated that any of R^{B} to R^{J} can be either the same or different.

For example, R^{B} to R^{J} independently can be H, a halogen (e.g., F, Cl, Br), a C₁-C₁₈ hydrocarbyl group, or a C₁-C₁₈ halogenated hydrocarbyl group, and the hydrocarbyl and halogenated hydrocarbyl groups, independently, can be C₁-C₁₂ groups, or C₁-C₁₀ groups, C₁-C₈ groups, C₁-C₆ groups, or C₁-C₄ groups. Any hydrocarbyl group, independently, can be an alkyl group, a cycloalkyl group, an aryl group (e.g., a phenyl group or a naphthyl group, optionally substituted), or an aralkyl group (e.g., a benzyl group, optionally substituted), and likewise for halogenated hydrocarbyl groups.

In an aspect, R^{B} to R^{J} independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). For instance, R^{B} to R^{E} independently can be H or a methyl group. Additionally or alternatively, R^{F} to R^{J} independently can be, in some aspects, H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group (—NO₂), a pentafluorophenyl group, or a trifluoromethyl group (CF₃). Additionally or alternatively, R^{D} and R^{E} can be joined to form a ring or ring system. Likewise, in some aspects, R^{F} and R^{G} can be joined to form a ring or ring system.

Each R^{A} in formulas (IIA) and (IIB) independently can be a C₁-C₁₈ hydrocarbyl group or a C₁-C₁₈ halogenated hydrocarbyl group, and any hydrocarbyl and halogenated hydrocarbyl options noted herein for R^{B} to R^{J} also can apply to R^{A}. Accordingly, in one aspect, each R^{A} independently can be a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group (one or more methyl substituents), a naphthyl group, a benzyl group, a pentafluorophenyl group, or a trifluoromethyl group (CF₃). In another aspect, each R^{A} independently can be a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

In formulas (IIA) and (IIB), Y can be O, NH, or CH₂. Thus, in one aspect, Y is O, while in another aspect, Y is NH, and in yet another aspect, Y is CH₂.

Referring now to the complex of formula (IIB), M can be Fe, Co, or Cr; m can be an oxidation state of M; and each Z independently can be H, a halogen, a C₁-C₁₈ hydrocarbyl group, or a C₁-C₁₈ halogenated hydrocarbyl group. The metal in formula (IIB), M, can be Fe, Co, or Cr, and thus in one aspect, for instance, M can be Fe or Cr, while in another aspect, M can be Fe; alternatively, M can be Co; or alternatively, M can be Cr.

Each Z in formula (IIB) independently can be any suitable monoanionic ligand, such as H, a halogen, a C₁-C₁₈ hydrocarbyl group, or a C₁-C₁₈ halogenated hydrocarbyl group, and any halogen, hydrocarbyl, and halogenated hydrocarbyl options noted herein for R^{B} to R^{J} also can apply to Z. Accordingly, in one aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a pentafluorophenyl group, or a trifluoromethyl group (CF₃). In another aspect, each Z independently can be H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a pentafluorophenyl group, or a trifluoromethyl group (CF₃). In yet another aspect, each Z can be Cl. In still another aspect, two or more Z can be joined to form a dianionic or trianionic or polyanionic ligand (e.g., catechol or a chelating group) to balance the oxidation state of M.

### Catalyst Compositions and Oligomerization Processes

Also encompassed herein are catalyst compositions. An illustrative catalyst composition can contain (a) any of the heteroatomic ligand transition metal compound complexes disclosed herein, and (b) an organoaluminum compound. Another illustrative catalyst composition can contain (A) any of the heteroatomic ligands disclosed herein, (B) any of the transition metal compounds disclosed herein, and (C) an organoaluminum compound. The catalyst composition can be prepared by contacting any of the aforementioned components in any order or sequence, at any suitable temperature and pressure, and in the presence or absence of an olefin (e.g., the olefin to be oligomerized, such as ethylene). If the catalyst composition is formed in a reactor at the time of contacting the olefin, then the appropriate pressures and temperatures will be those that are typical of the oligomerization process, discussed further below.

In an aspect, the heteroatomic ligand (compound) utilized in the catalyst composition and oligomerization process can be Ligand 5, Ligand 7, Ligand 9, or Ligand 11, or a mixture or combination thereof, as shown below in Table 1. Thus, for example, the heteroatomic ligand (compound) utilized in the catalyst composition and oligomerization process can be Ligand 5; alternatively, Ligand 7; alternatively, Ligand 9; or alternatively, Ligand 11.

In another aspect, the heteroatomic ligand (compound) utilized in the catalyst composition and oligomerization process can be Ligand 6, Ligand 8, Ligand 10, Ligand 12, Ligand 13, or Ligand 15, or a mixture or combination thereof, as shown below in Table 1. Thus, for example, the heteroatomic ligand (compound) utilized in the catalyst composition and oligomerization process can be Ligand 6; alternatively, Ligand 8; alternatively, Ligand 10; alternatively, Ligand 12; alternatively, Ligand 13; or alternatively, Ligand 15.

Often, the catalyst composition can further contain a hydrocarbon diluent (or solvent). For instance, the hydrocarbon diluent (or solvent) can comprise any suitable saturated aliphatic hydrocarbon, any suitable aromatic hydrocarbon, or any combination thereof. The saturated aliphatic hydrocarbon can be a linear aliphatic hydrocarbon, a branched aliphatic hydrocarbon, or a cyclic aliphatic hydrocarbon, as well as combinations thereof. Thus, the hydrocarbon diluent (or solvent) can comprise a linear alkane, a branched alkane, a cyclic alkane, or a combination thereof. Illustrative examples of saturated aliphatic hydrocarbons that can be utilized as the diluent (or solvent), either singly or in combination, include propane, butane (e.g., n-butane or isobutane), pentane (e.g., n-pentane, neopentane, cyclopentane, or isopentane), hexane, heptane, octane, cyclohexane, methyl cyclohexane, and the like, as well combinations thereof. In a particular aspect of this disclosure, the hydrocarbon diluent (or solvent) can comprise (or consist essentially of, or consist of) cyclohexane.

Additionally or alternatively, the hydrocarbon diluent (or solvent) can comprise an aromatic hydrocarbon, such as benzene, toluene, ethylbenzene, xylene, styrene, mesitylene, and the like. Combinations of two or more aromatic hydrocarbons can be utilized, if desired.

The relative amount of the organoaluminum compound versus that of the heteroatomic ligand transition metal compound complex (or versus that of the heteroatomic ligand and the transition metal compound) is not particularly limited. Nonetheless, molar ratios of Al:transition metal (for example, Al:Cr or Al:Fe) or Al:ligand in the catalyst composition can range from 10:1 to 5,000:1, such as from 50:1 to 3,000:1, from 50:1 to 3,000:1, from 75:1 to 2,000:1, from 100:1 to 2,000:1, or from 100:1 to 1,000:1, and the like. If, for example, more than one transition metal (or ligand) and/or more than one organoaluminum are employed, these ratios are based on the total moles of respective transition metals, ligands, and organoaluminums.

When both a heteroatomic ligand and a transition metal compound are present, the molar ratio of ligand:transition metal often ranges from 10:1 to 1:10, and more often, from 8:1 to 1:8, from 5:1 to 1:5, from 4:1 to 1:4, or from 2:1 to 1:2, and the like. In some aspects, the transition metal compound is present in a molar excess relative to the heteroatomic ligand, although this is not a requirement.

Generally, the transition metal compound or the transition metal compound of the heteroatomic ligand transition metal compound complexes described herein can have the formula M(X¹)ₚ. In this formula, M can be Fe, Co, or Cr, and p is an oxidation state of M. Each X¹ independently can be any suitable monoanionic ligand. Typically, the transition metal atom of the transition metal compound can have any positive oxidation state available to the transition metal atom. For instance, the cobalt atom can have an oxidation state of from +2, while the iron atom or the chromium atom can have various oxidation states, including +2 and +3.

The monoanionic ligand (X¹) can be a halogen (e.g., fluorine or chlorine), a carboxylate, a β-diketonate, a hydrocarboxide, a nitrate, or a chlorate. The hydrocarboxide can be an alkoxide, an aryloxide, or an aralkoxide. Generally, any carboxylate of the transition metal compound independently can be a C₁ to C₂₀ carboxylate, or alternatively, a C₁ to C₁₀ carboxylate. In an aspect, each carboxylate independently can be acetate, a propionate, a butyrate, a pentanoate, a hexanoate, a heptanoate, an octanoate, a nonanoate, a decanoate, an undecanoate, or a dodecanoate; or alternatively, a pentanoate, a hexanoate, a heptanoate, an octanoate, a nonanoate, a decanoate, an undecanoate, or a dodecanoate. In some aspects, each carboxylate independently can be acetate, propionate, n-butyrate, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, capronate (n-hexanoate); alternatively, n-heptanoate; alternatively, caprylate (n-octanoate); or alternatively, 2-ethylhexanoate. In some aspects, the carboxylate can be triflate (trifluoroacetate). In other aspects, two or more X¹ can be joined to form a dianionic or trianionic or polyanionic ligand (e.g., catechol or a chelating group) to balance the oxidation state of M.

Generally, each β-diketonate of the transition metal compound independently can be any C₁ to C₂₀ a β-diketonate; or alternatively, any C₁ to C₁₀ β-diketonate. In an aspect, each β-diketonate independently can be acetylacetonate (i.e., 2,4-pentanedionate), hexafluoroacetylacetonate (i.e., 1,1,1,5,5,5-hexafluoro-2,4-pentanedionate), or benzoylacetonate; alternatively, acetylacetonate; alternatively, hexafluoroacetylacetonate; or alternatively, benzoylacetonate.

Generally, each hydrocarboxide of the transition metal compound independently can be any C₁ to C₂₀ hydrocarboxide; or alternatively, any C₁ to C₁₀ hydrocarboxide. In an aspect, each hydrocarboxide independently can be a C₁ to C₂₀ alkoxide; alternatively, a C₁ to C₁₀ alkoxide; alternatively, a C₆ to C₂₀ aryloxide; or alternatively, a C₆ to C₁₀ aryloxide. In an aspect, each alkoxide independently can be methoxide, ethoxide, a propoxide, or a butoxide; alternatively, methoxide, ethoxide, isopropoxide, or tert-butoxide; alternatively, methoxide; alternatively, an ethoxide; alternatively, an iso-propoxide; or alternatively, a tert-butoxide. In an aspect, the aryloxide can be phenoxide.

In some non-limiting aspects, the transition metal compound and/or the transition metal compound of the heteroatomic ligand transition metal compound complex can comprise, can consist essentially of, or consist of, a cobalt (II) halide, a chromium (II) halide, an iron (II) halide, a cobalt (II) carboxylate, a chromium (II) carboxylate, an iron (II) carboxylate, a cobalt (II) β-diketonate, a chromium (II) β-diketonate, or an iron (II) β-diketonate; alternatively, a chromium (III) halide, an iron (III) halide, a chromium (III) carboxylate, an iron (III) carboxylate, a chromium (III) β-diketonate, or an iron (III) β-diketonate; alternatively, cobalt (II) nitrate, chromium (II) nitrate, iron (II) nitrate, chromium (III) nitrate, or iron (III) nitrate; or alternatively, cobalt (II) acetylacetonate, chromium (II) acetylacetonate, iron (II) acetylacetonate, chromium (III) acetylacetonate, or iron (III) acetylacetonate.

While not shown in the transition metal compound names and formulas and/or heteroatomic ligand transition metal compound complex formulas and structures provided herein, one of ordinary skill in the art will recognize that a neutral ligand, Q, can be associated with the transition metal compounds and/or the heteroatomic ligand transition metal compound complexes described/depicted herein which do not explicitly disclose/depict a neutral ligand. Consequently, transition metal compounds and/or heteroatomic ligand transition metal compound complexes having a neutral ligand, Q, can be considered as equivalent to the transition metal compounds and/or heteroatomic ligand transition metal compound complexes depicted herein not having the neutral ligand, Q. Additionally, it should be understood that while some of the transition metal compounds and/or heteroatomic ligand transition metal compound complexes described/depicted/provided herein do not formally show the presence of a neutral ligand, the transition metal compounds and/or heteroatomic ligand transition metal compound complexes having neutral ligands (e.g., nitriles and ethers, among others) are fully contemplated and encompassed herein as potential transition metal compounds and/or heteroatomic ligand transition metal compound complexes that can be utilized in the catalyst system used in aspects of the present disclosure.

Generally, the neutral ligand of any transition metal compound and/or heteroatomic ligand transition metal compound complex, when present, independently can be any neutral ligand that forms an isolatable compound with the transition metal compound and/or heteroatomic ligand transition metal compound complex. In an aspect, each neutral ligand independently can be a nitrile or an ether; alternatively, a nitrile; or alternatively, an ether. The number of neutral ligands, q, can be any number that forms an isolatable compound with the transition metal compound, and/or heteroatomic ligand transition metal compound complex. In an aspect, the number of neutral ligands can be from 0 to 6; alternatively, 0 to 3; alternatively, 0; alternatively, 1; alternatively, 2; alternatively, 3; or alternatively, 4.

Generally, each nitrile ligand independently can be a C₂ to C₂₀ nitrile; or alternatively, a C₂ to C₁₀ nitrile. In an aspect, each nitrile independently can be acetonitrile, propionitrile, a butyronitrile, benzonitrile, or any combination thereof; alternatively, acetonitrile; alternatively, propionitrile; alternatively, a butyronitrile; or alternatively, benzonitrile.

Generally, each ether ligand independently can be a C₂ to C₄₀ ether; alternatively, a C₂ to C₃₀ ether; or alternatively, a C₂ to C₂₀ ether. In some aspects, each ether ligand independently can be dimethyl ether, diethyl ether, a dipropyl ether, a dibutyl ether, methyl ethyl ether, a methyl propyl ether, a methyl butyl ether, tetrahydrofuran, a dihydrofuran, 1,3-dioxolane, tetrahydropyran, a dihydropyran, a pyran, a dioxane, furan, benzofuran, isobenzofuran, dibenzofuran, diphenyl ether, a ditolyl ether, or any combination thereof; alternatively, dimethyl ether, diethyl ether, a dipropyl ether, a dibutyl ether, methyl ethyl ether, a methyl propyl ether, a methyl butyl ether, or any combination thereof; tetrahydrofuran, a dihydrofuran, 1,3-dioxolane, tetrahydropyran, a dihydropyran, a pyran, a dioxane, or any combination thereof; furan, benzofuran, isobenzofuran, dibenzofuran, or any combination thereof; diphenyl ether, a ditolyl ether, or any combination thereof; alternatively, dimethyl ether; alternatively, diethyl ether; alternatively, a dipropyl ether; alternatively, a dibutyl ether; alternatively, methyl ethyl ether; alternatively, a methyl propyl ether; alternatively, a methyl butyl ether; alternatively, tetrahydrofuran; alternatively, a dihydrofuran; alternatively, 1,3-dioxolane; alternatively, tetrahydropyran; alternatively, a dihydropyran; alternatively, a pyran; alternatively, a dioxane; alternatively, furan; alternatively, benzofuran; alternatively, isobenzofuran; alternatively, dibenzofuran; alternatively, diphenyl ether; or alternatively, a ditolyl ether.

While the heteroatomic ligand transition metal compound complex formulas and structures provided herein are shown as neutral complexes, one of ordinary skill in the art will recognize that heteroatomic ligand transition metal compound complexes can comprise or can exist as “ate” complexes comprising a negatively charged heteroatomic ligand transition metal compound complex and an associated positively charged metal or metal complex cation. Additionally, it should be understood that while the heteroatomic ligand transition metal compound complexes described/depicted/provided herein are shown as neutral complexes, the “ate” complexes comprising a negatively charged heteroatomic ligand transition metal compound complex and an associated positively charged metal or metal complex cation are implicitly and fully contemplated as potential heteroatomic ligand transition metal compound complexes that can be utilized in the catalyst system used in aspects of the present disclosure.

Generally, the organoaluminum compound utilized in the catalyst systems disclosed herein can be any organoaluminum compound which in conjunction with the heteroatomic ligand transition metal compound complex (or the transition metal compound and heteroatomic ligand) can catalyze the formation of an oligomer product. In an aspect, the organoaluminum compound can comprise, can consist essentially of, or can consist of, an aluminoxane, an alkylaluminum compound, or any combination thereof; alternatively, an aluminoxane; or alternatively, an alkylaluminum compound. In an aspect, the alkylaluminum compound can comprise, can consist essentially of, or can consist of, a trialkylaluminum, an alkylaluminum halide, an alkylaluminum alkoxide, or any combination thereof. In some aspects, the alkylaluminum compound can comprise, can consist essentially of, or can consist of, a trialkylaluminum, an alkylaluminum halide, or any combination thereof; alternatively, a trialkylaluminum, an alkylaluminum alkoxide, or any combination thereof; or alternatively, a trialkylaluminum. In other aspects, the alkylaluminum compound can be a trialkylaluminum; alternatively, an alkylaluminum halide; or alternatively, an alkylaluminum alkoxide. In an aspect, the aluminoxane utilized in the catalyst systems can comprise, can consist essentially of, or can consist of, any aluminoxane which in conjunction with the heteroatomic ligand transition metal compound complex (or the transition metal compound and heteroatomic ligand) can catalyze the formation of an oligomer product. In a non-limiting aspect, the aluminoxane can have a repeating unit characterized by the Formula (III):

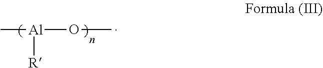

In formula (III), R′ is a linear or branched alkyl group. Alkyl groups of the aluminoxanes and alkylaluminum compounds are independently described herein and can be utilized without limitation to further describe the aluminoxanes having Formula (III) and/or the alkylaluminum compounds. Generally, n of Formula (III) can be greater than 1; or alternatively, greater than 2. In an aspect, n can range from 2 to 15; or alternatively, range from 3 to 10.

In an aspect, each halide of any alkylaluminum halide disclosed herein can independently be fluoride, chloride, bromide, or iodide; or alternatively, chloride, bromide, or iodide. In an aspect, each halide of any alkylaluminum halide disclosed herein can be fluoride; alternatively, chloride; alternatively, bromide; or alternatively, iodide.

In an aspect, each alkyl group of an aluminoxane and/or alkylaluminum compound independently can be a C₁ to C₂₀ alkyl group; alternatively, a C₁ to C₁₀ alkyl group; or alternatively, a C₁ to C₆ alkyl group. In an aspect, each alkyl group of an aluminoxane and/or alkylaluminum compound independently can be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some aspects, each alkyl group of an aluminoxane and/or alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

In an aspect, each alkoxide group of any alkylaluminum alkoxide disclosed herein independently can be a C₁ to C₂₀ alkoxy group, a C₁ to C₁₀ alkoxy group, or a C₁ to C₆ alkoxy group. In an aspect, each alkoxide group of any alkylaluminum alkoxide disclosed herein independently can be a methoxy group, an ethoxy group, a propoxy group, a butoxy group, a pentoxy group, a hexoxy group, a heptoxy group, or an octoxy group; alternatively, a methoxy group, an ethoxy group, a butoxy group, a hexoxy group, or an octoxy group. In some aspects, each alkoxide group of any alkylaluminum alkoxide disclosed herein independently can be a methoxy group, an ethoxy group, an n-propoxy group, an n-butoxy group, an iso-butoxy group, an n-hexoxy group, or an n-octoxy group; alternatively, a methoxy group, an ethoxy group, an n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, an n-hexoxy group; or alternatively, an n-octoxy group.

In a non-limiting aspect, useful trialkylaluminum compounds can include trimethylaluminum, tricthylaluminum, tripropylaluminum, tributylaluminum, trihexylaluminum, trioctylaluminum, or mixtures thereof. In some non-limiting aspects, useful trialkylaluminum compounds can include trimethylaluminum, tricthylaluminum, tripropylaluminum, tri-n-butylaluminum, tri-isobutylaluminum, trihexylaluminum, tri-n-octylaluminum, or mixtures thereof; alternatively, triethylaluminum, tri-n-butylaluminum, tri-isobutylaluminum, trihexylaluminum, tri-n-octylaluminum, or mixtures thereof; alternatively, triethylaluminum, tri-n-butylaluminum, trihexylaluminum, tri-n-octylaluminum, or mixtures thereof. In other non-limiting aspects, useful trialkylaluminum compounds can include trimethylaluminum; alternatively, triethylaluminum; alternatively, tripropylaluminum; alternatively, tri-n-butylaluminum; alternatively, tri-isobutylaluminum; alternatively, trihexylaluminum; or alternatively, tri-n-octylaluminum.

In a non-limiting aspect, useful alkylaluminum halides can include diethylaluminum chloride, diethylaluminum bromide, ethylaluminum dichloride, ethylaluminum sesquichloride, and mixtures thereof. In some non-limiting aspects, useful alkylaluminum halides can include diethylaluminum chloride, ethylaluminum dichloride, ethylaluminum sesquichloride, and mixtures thereof. In other non-limiting aspects, useful alkylaluminum halides can include diethylaluminum chloride; alternatively, diethylaluminum bromide; alternatively, ethylaluminum dichloride; or alternatively, ethylaluminum sesquichloride.

In a non-limiting aspect, the aluminoxane can comprise, consist essentially of, or consist of, methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-entylaluminoxane, 3-pentyl-aluminoxane, iso-pentyl-aluminoxane, neopentylaluminoxane, or mixtures thereof. In some non-limiting aspects, the aluminoxane can comprise, consist essentially of, or consist of, methylaluminoxane (MAO), modified methylaluminoxane (MMAO), isobutyl aluminoxane, t-butyl aluminoxane, or mixtures thereof. In other non-limiting aspects, the aluminoxane can comprise, consist essentially of, or consist of, methylaluminoxane (MAO); alternatively, ethylaluminoxane; alternatively, modified methylaluminoxane (MMAO); alternatively, n-propylaluminoxane; alternatively, iso-propyl-aluminoxane; alternatively, n-butylaluminoxane; alternatively, sec-butylaluminoxane; alternatively, iso-butylaluminoxane; alternatively, t-butyl aluminoxane; alternatively, 1-pentyl-aluminoxane; alternatively, 2-pentylaluminoxane; alternatively, 3-pentyl-aluminoxane; alternatively, iso-pentyl-aluminoxane; or alternatively, neopentylaluminoxane.

Also encompassed herein are oligomerization processes. For instance, an oligomerization process consistent with aspects of this invention can comprise (i) contacting ethylene, any of the catalyst compositions disclosed herein, an organic reaction medium, and optionally hydrogen, in an oligomerization reactor, (ii) forming an oligomer product in the oligomerization reactor, the oligomer product comprising hexenes and octenes, and (iii) discharging an effluent stream from the oligomerization reactor, the effluent stream comprising unreacted ethylene and the oligomer product.

Among other constituents, the effluent stream contains an oligomer product, which can comprise hexenes and octenes, as well as other C++ linear alpha olefins. The amount of octenes in the oligomer product typically can fall within a range from 5 to 99 wt. %, based on the total amount of oligomers in the oligomer product. In an aspect, the minimum amount of octenes in the oligomer product can be 5, 10, 20, 30 or 40 wt. %. In another aspect, the maximum amount of octenes in the oligomer product can be 99, 95, 92.5, 90, 87.5, or 85 wt. %. Generally, the amount of octenes in the oligomer product can range from any minimum amount of octenes in the oligomer product to any maximum amount of octenes in the oligomer product described herein. For instance, the amount of octenes—based on the total weight of oligomers in the oligomer product—can be from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes.

Additionally or alternatively, the oligomer product can contain any suitable amount of hexenes. In an aspect, the minimum amount of hexenes in the oligomer product can be 15, 20, 25, 30, or 35 wt. %. In another aspect, the maximum amount of hexenes in the oligomer product can be 75, 65, 60, 55, or 50 wt. %. Generally, the amount of hexenes in the oligomer product can range from any minimum amount of hexenes in the oligomer product to any maximum amount of hexenes in the oligomer product described herein. For instance, the amount of hexenes—based on the total weight of oligomers in the oligomer product—can be from 20 to 60 wt. %, from 25 to 55 wt. %, or from 30 to 50 wt. % hexenes.

The amount of conversion of ethylene in the oligomerization reactor is not particularly limited, and generally the minimum ethylene conversion can be at least 20, 30, 35, 40, 45, or 50 wt. %, while the maximum ethylene conversion can be 99, 95, 90, 80, 75, 70, or 65 wt. %. Generally, the ethylene conversion in the reactor can range from any minimum conversion to any maximum conversion described herein. For instance, the ethylene conversion can range from 20 to 95 wt. %, from 30 to 90 wt. %, from 40 to 80 wt. %, from 50 to 70 wt. %, or from 55 to 65 wt. %. The ethylene conversion is based on the amount of ethylene entering the reactor and the amount of (unreacted)ethylene in the effluent stream.

Referring now to the step of contacting ethylene, the catalyst composition, the organic reaction medium, and optionally hydrogen, in the oligomerization reactor. Hydrogen use is optional in this step, thus in one aspect, hydrogen is not present in this step of the process, while in another aspect, hydrogen is present in this step of the process.

Ethylene, the catalyst composition, the organic reaction medium, and hydrogen can be combined in any order or sequence and introduced into the oligomerization reactor separately or in any combination. For instance, hydrogen and ethylene can be combined and fed to the reactor separately from the catalyst composition. This invention is not limited by the manner in which the respective feed streams are introduced into the reactor. In one aspect, for instance, the catalyst composition can be formed first and then introduced into the oligomerization reactor. In this aspect, the organoaluminum compound is contacted with the heteroatomic ligand transition metal compound complex, or with the heteroatomic ligand and the transition metal compound, prior to being introduced into the reactor. In another aspect, however, the catalyst composition can be formed within the oligomerization reactor. In this aspect, the organoaluminum compound and the heteroatomic ligand transition metal compound complex (or the heteroatomic ligand and the transition metal compound) are introduced separately into the reactor, and the catalyst composition is formed within the reactor.

Any suitable organic reaction medium can be used in the disclosed oligomerization processes, such as a hydrocarbon. Illustrative hydrocarbons can include, for example, saturated aliphatic hydrocarbons, aromatic hydrocarbons, and the like, as well as combinations thereof. The organic reaction medium can be selected from the same materials as that for the hydrocarbon diluent (or solvent) in the catalyst compositions. Thus, the organic reaction medium can comprise any alkane or aromatic hydrocarbon disclosed herein, as well as any combination thereof. While not required, the organic reaction medium can comprise the same material as that of the hydrocarbon diluent or solvent. Further, in a particular aspect of this disclosure, the organic reaction medium can comprise (or consist essentially of, or consist of) cyclohexane.

Forming the oligomer product in the oligomerization reactor can be accomplished at any suitable oligomerization temperature and pressure. Often, the oligomer product can be formed at a minimum temperature of 0° C., 20° C., 30° C., 40° C., 45° C., or 50° C.; additionally or alternatively, at a maximum temperature of 165° C., 160° C., 150° C., 140° C., 130° C., 115° C., 100° C., or 90° C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 20 to 160, from 20 to 115, from 40 to 160, from 40 to 140, from 50 to 150, from 50 to 140, from 50 to 130, from 50 to 100, from 60 to 115, from 70 to 100, or from 75 to 95° C. Other appropriate oligomerization temperatures and temperature ranges are readily apparent from this disclosure.

The oligomer product can be formed at a minimum pressure (or ethylene partial pressure) of 50 psig (344 kPa), 100 psig (689 kPa), 200 psig (1.4 MPa), or 250 psig (1.5 MPa); additionally or alternatively, at a maximum pressure (or ethylene partial pressure) of 4,000 psig (27.6 MPa), 3,000 psig (20.9 MPa), 2,000 psig (13.8 MPa), or 1,500 psig (10.3 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 50 psig (344 kPa) to 4,000 psig (27.6 MPa), from 100 psig (689 kPa) to 3,000 psig (20.9 MPa), from 100 psig (689 kPa) to 2,000 psig (13.8 MPa), from 200 psig (1.4 MPa) to 2,000 psig (13.8 MPa), from 200 psig (1.4 MPa) to 1,500 psig (10.3 MPa), or from 250 psig (1.5 MPa) to 1,500 psig (10.3 MPa). Other appropriate oligomerization pressures (or ethylene partial pressures) are readily apparent from this disclosure.

When used, hydrogen can be fed directly to the reactor, or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

The oligomerization reactor in which the oligomer product is formed can comprise any suitable reactor. Non-limiting examples of reactor types can include a stirred tank reactor, a plug flow reactor, or any combination thereof; alternatively, a fixed bed reactor, a continuous stirred tank reactor, a loop reactor, a solution reactor, a tubular reactor, a recycle reactor, or any combination thereof. In an aspect, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

Disclosed below are methods for preparing heteroatomic ligands that are utilized in the preparation of the heteroatomic ligand transition metal compound complexes and catalyst compositions of Examples 1-21. The heteroatomic ligand transition metal compound complexes are prepared by combining the heteroatomic ligands with a transition metal compound (e.g., a soluble iron source). Advantageously, the inclusion of the disclosed heteroatomic ligand transition metal compound complexes in catalyst compositions leads to catalyst systems that exhibit high catalyst productivities and C₄-C₂₆ oligomer product selectivity. Additionally, the preparation methods of intermediate compounds necessary for the synthesis of the heteroatomic ligands are disclosed below.

For ¹H NMR data and ³¹P NMR data, the general procedure utilized a Bruker Instrument (e.g., Av400x) and approximately 3-10 mg of the respective compound dissolved in an appropriate NMR solvent (e.g., C₆D₆ and/or CDCl₃).

### SYNTHESIS OF INTERMEDIATE COMPOUNDS

Intermediate C (1-(6-aminopyridin-2-yl) ethan-1-one) was prepared as follows. A solution of 6-aminopicolinonitrile was prepared (1 g, 8.4 mmol) in THF (20 mL), stirred at 0° C. for 15 min, and MeMgBr (14 mL, 42.0 mmol) was added dropwise. The reaction was stirred for 4 hr at 25° C. and then quenched with NH₄Cl and extracted with EtOAc. The solution was rinsed with brine and dried with Na₂SO₄. The volatiles were removed in vacuo. The Intermediate C compound was obtained as a brown solid which turned yellow upon scraping with a spatula (1.12 g, 96% yield). ¹H NMR (500 MHZ, CDCl₃), δ=7.56 (t, J=7.8, 1H), 7.4 (d, J=7.4, 1H), 6.67 (d, J=8.2, 1H), 4.53 (brs, 2H), 2.63 (s, 3H). The chemical structure of Intermediate C is shown below:

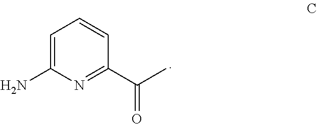

Intermediate D (6-(1-(mesitylimino)ethyl)pyridin-2-amine) was prepared as follows. In a round bottom flask, a solution of 1-(6-aminopyridin-2-yl) ethan-1-one (1 g, 7.35 mmol) was added to 2,4,6-trimethylaniline (1.55 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (1:1 hexane to EtOAc with 1% NEt₃) to yield the Intermediate D compound (0.5505 g, 31.8% yield). ¹H NMR (500 MHz, CDCl₃) δ=7.66 (d, J=7.5, 1H), 7.54 (t, J=7.8, 1H), 6.86 (s, 2H), 6.58 (d, J=7.7, 1H), 4.46 (brs, 2H), 2.28 (s, 3H), 2.08 (s, 3H), 1.99 (s, 6H). The chemical structure of Intermediate D is shown below:

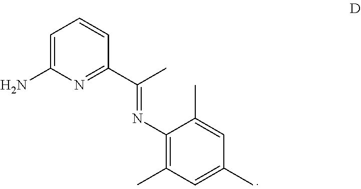 [SMILES: C/C(=N\c1c(C)cc(C)cc1C)c1cccc(N)n1]

Intermediate E (6-(1-((2,4-dimethylphenyl)imino)ethyl)pyridin-2-amine) was prepared as follows. In a round bottom flask, a solution of 1-(6-aminopyridin-2-yl) ethan-1-one (1 g, 7.35 mmol) was added to 2,4-dimethylaniline (1.35 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (5:1 hexane to EtOAc with 1% NEt₃) to yield the Intermediate E compound (117 mg, 6.6% yield). ¹H NMR (500 MHZ, CDCl₃) δ=7.62-7.60 (d, J=7.53, 1H), 7.55-7.52 (t, J=7.92, 1H), 7.02 (s, 1H), 6.59-6.57 (d, J=8.15, 1H), 6.56-6.54 (d, J=7.91, 1H), 4.45 (s, 2H), 2.31 (s, 3H), 2.18 (s, 3H), 2.06 (s, 3H). The chemical structure of Intermediate E is shown below:

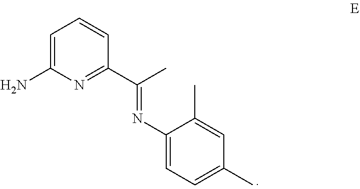 [SMILES: C/C(=N\c1ccc(C)cc1C)c1cccc(N)n1]

Intermediate F (6-(1-((2,4-dimethylphenyl)imino)ethyl)pyridin-2-amine) was prepared as follows. In a round bottom flask, a solution of 1-(6-aminopyridin-2-yl) ethan-1-one (1 g, 7.35 mmol) was added to 2,6-methylaniline (1.35 mL, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo and the residues were purified via flash chromatography (1:1 hexane to EtOAc in 1% NEt₃) to yield the Intermediate F compound (0.430 g, 56% yield). ¹H NMR (500 MHZ, CDCl₃), δ=7.68-7.66 (d, J=7.88, 1H), 7.57-7.54 (t, J=7.88, 1H), 7.05-7.03 (d, J=6.76, 1H), 6.93-6.90 (t, J=7.32, 1H), 6.61-6.59 (d, J=8.45, 1H), 4.51 (s, 2H), 2.09 (s, 3H), 2.02 (s, 6H). The chemical structure of Intermediate F is shown below:

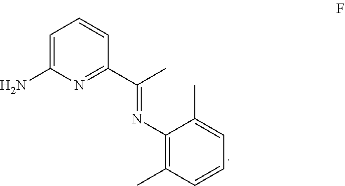 [SMILES: C/C(=N\c1c(C)cccc1C)c1cccc(N)n1]

Intermediate G (3-(1-(o-tolylimino)ethyl) aniline) was prepared as follows. In a round bottom flask, a solution of 1-(6-aminopyridin-2-yl) ethan-1-one (1 g, 7.35 mmol) was added to 2-methylaniline (1.18 g, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo. The residues were purified via flash chromatography (3:1 hexane:EtOAc in 1% NEt₃) to yield the Intermediate G compound (0.148 g, 9.0% yield). ¹H NMR (500 MHZ, CDCl₃) δ=7.65-7.63 (m, 1H), 7.57-7.53 (m, 1H), 7.39 (s, 1H), 7.36-7.29 (m, 2H), 6.75-6.73 (d, J=8.08, 1H), 6.69-6.67 (d, J=8.08, 1H), 6.60-6.58 (d, J=8.08, 1H), 4.47 (s, 2H), 2.24 (s, 3H), 2.14 (s, 3H). The chemical structure of Intermediate G is shown below:

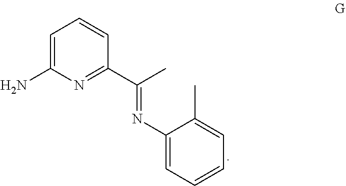 [SMILES: C/C(=N\c1ccccc1C)c1cccc(N)n1]

Intermediate H (6-(1-(p-tolylimino)ethyl)pyridin-2-amine) was prepared as follows. In a round bottom flask, a solution of 1-(6-aminopyridin-2-yl) ethan-1-one (1 g, 7.35 mmol) was added to 4-methylaniline (1.18 g, 11.017 mmol), p-TsOH (14 mg, 0.073 mmol), and n-butanol (25 mL). The resulting solution was purged with N₂ and refluxed for 24 hr using azeotropic removal of water with a Dean-Stark trap. The volatiles were removed in vacuo and the residues were purified via flash chromatography (5:1 hexane:EtOAc in 1% NEt₃) to yield the Intermediate H compound (0.3961 g, 23.9% yield). ¹H NMR (500 MHZ, CDCl₃), δ=7.55-7.51 (m, 2H), 7.16-7.14 (d, J=8.65, 2H), 6.72-6.70 (d, J=8.07, 2H), 6.58-6.56 (dt, J=7.00, 1H), 4.48 (s, 2H), 2.35 (s, 3H), 2.26 (s, 3H). The chemical structure of Intermediate H is shown below:

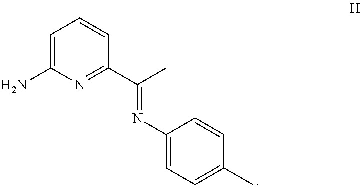 [SMILES: C/C(=N\c1ccc(C)cc1)c1cccc(N)n1]

The Intermediate I, J, K, L, and M compounds were synthesized by the following reaction pathway:

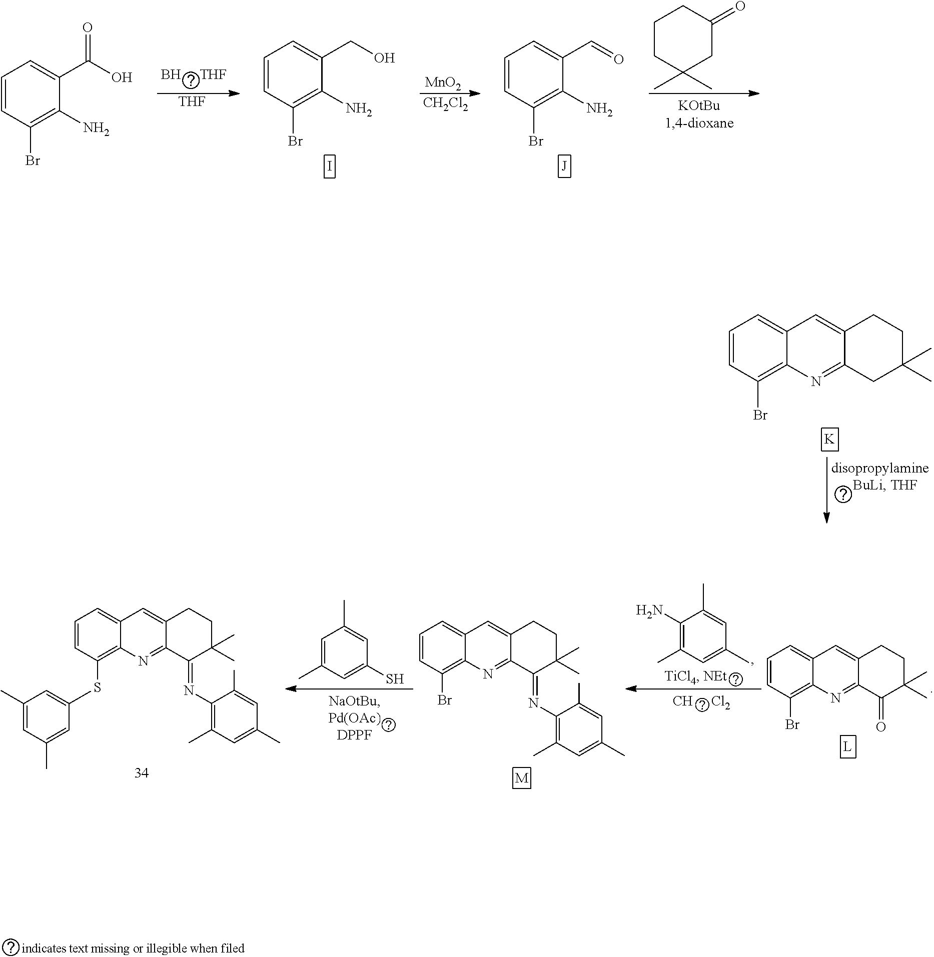

Intermediate I (2-amino-3-bromophenyl) methanol) was prepared as follows. A solution of 2-amino-3-bromobenzoic acid (3 g, 13.9 mmol, 1 equiv.) was prepared in THF (12.5 mL) and stirred at 0° C. BH₃THF (1.0M in THF) (20.8 mL, 1.5 equiv.) was added to the solution dropwise and refluxed for 16 hr. The mixture was filtered and the volatiles were removed in vacuo. The product was purified via flash column chromatography (petroleum ether:ethyl acetate 1:1) to obtain the Intermediate I compound as a white solid (1.9 g, 69% yield). ¹H NMR (500 MHZ, CDCl₃), δ=7.41-7.39 (dd, J=1.5, 8.1, 1H), 7.05-7.00 (d, J=7.5, 1H), 6.59-6.55 (t, J=7.8, 1H), 4.73 (brs, 2H), 4.69 (s, 2H).

Intermediate J (2-amino-3-bromobenzaldehyde) was prepared as follows. MnO₂ (1.05 g, 12 mmol, 4 equiv.) was added to a solution of Intermediate I (2-amino-3-bromophenyl) methanol) (610 mg, 3 mmol, 1 equiv.) in CH₂Cl₂ (15.3 mL) and stirred at room temperature for 24 hr. The reaction mixture was filtered, and the volatiles removed in vacuo to obtain the Intermediate J compound (0.572 g, 95% yield). ¹H NMR (500 MHZ, CDCl₃), δ=9.83 (s, 1H), 7.63-7.61 (dd, J=1.4, 7.9, 1H), 7.49-7.47 (dd, J=1.2, 7.7, 1H), 6.69-6.22 (t, J=7.8, 1H), 6.67 (brs, 2H).

Intermediate K (5-bromo-3,3-dimethyl-1,2,3,4-tetrahydroacridine) was prepared as follows. A solution of Intermediate J (2-amino-3-bromobenzaldehyde) (1.35 g, 6.8 mmol, 1 equiv.) was added to 3,3-dimethylcyclohexan-1-one (1 g, 8.1 mmol, 1.2 equiv.), KOtBu (1.8 g, 16 mmol, 2.4 equiv.), and 1,4-dioxane (34 mL), and refluxed for 1 hr. The reaction mixture was concentrated, rinsed with Et₂O, and filtered. Purification by column chromatography (hexanes:EtOAc 10:1) yielded the Intermediate K compound as a yellow solid (1.8 g, 91% yield). ¹H NMR (500 MHZ, CDCl₃), δ=7.95-7.93 (dd, J=1.1, 7.5, 1H), 7.84 (s, 1H), 7.69-7.67 (d, J=8.2, 1H), 7.29-7.26 (t, J=7.9, 1H), 3.06-3.03 (t, J=6.9, 2H), 3.00 (s, 2H), 1.71-1.69 (t, J=6.9, 2H), 1.08 (s, 6H).

Intermediate L (5-bromo-3,3-dimethyl-2,3-dihydroacridin-4 (1H)-one) was prepared as follows. Under an inert atmosphere, diisopropylamine (0.58 mL, 4.3 mmol, 2.5 equiv.) was cooled to −15° C. and n-butyllithium (1.38 mL, 3.4 mmol, 2 equiv.) was added dropwise. The reaction was stirred for 15 min and then THF was added (10 mL). The reaction was warmed to room temperature and stirred for 1 hr. The reaction was then cooled to −15° C. and a solution of Intermediate K (5-bromo-3,3-dimethyl-1,2,3,4-tetrahydroacridine) (0.500 g, 1.7 mmol, 1 equiv.) in THF (15 mL) was slowly added. The reaction was stirred at room temperature for 3 hr. The reaction was then cooled to −15° C. and isoamyl nitrate (2.3 mL, 11.7 mmol, 10 equiv.) was added and stirred at room temperature for 4 hr. The reaction was quenched with H₂O, and extracted with DCM, then concentrated and HCl (18 mL) was added and refluxed overnight. The mixture was neutralized with a NaOH solution. The reaction was extracted with DCM. The volatiles were removed in vacuo and purified by column chromatography (3:1 PE:EtOAc) to yield the Intermediate L compound (0.315 g, 60% yield). ¹H NMR (500 MHz, CDCl₃), δ=8.11 (s, 1H), 8.07-8.05 (dd, J=1.0, 7.5, 1H), 7.77-7.76 (d, J=8.5, 1H), 7.46-7.42 (t, J=8.0, 1H), 3.25-3.22 (t, J=6.6, 2H), 2.12-2.10 (t, J=6.5, 2H), 1.34 (s, 6H).

Intermediate M (E)-5-bromo-N-mesityl-3,3-dimethyl-2,3-dihydroacridin-4 (1H)-imine) was prepared as follows. A solution of Intermediate L (5-bromo-3,3-dimethyl-2,3-dihydroacridin-4 (1H)-one) (300 mg, 0.98 mmol, 1 equiv.) in CH₂Cl₂ (10 mL) was prepared and distilled TiCl₄ (0.11 mL, 0.98 mmol, 1 equiv.) was added to the solution at 0° C. and stirred for 10 min. At 0° C., 2,4,6-trimethylaniline (0.300 g, 0.98 mmol, 1 equiv.) was added to the solution (0.98 mmol, 1 equiv.) and stirred for 10 min, followed by addition of triethylamine (0.1 mL). The reaction was warmed to room temperature and stirred overnight. The reaction mixture was quenched with NH₄Cl and extracted with DCM and washed with brine. The volatiles were then removed in vacuo. The product was then purified via flash chromatography (petroleum ether:ethyl acetate, 50:1) to yield the Intermediate M compound (0.294 g, 71% yield). ¹H NMR (500 MHZ, CDCl₃), δ 7.93 (s, 1H), 7.81-7.79 (d, J=7.5, 1H), 7.62-7.60 (dd, J=1.0, 8.3, 1H), 7.26-7.24 (t, J=7.7, 1H), 6.71 (s, 2H), 3.14-3.11 (t, J=6.6, 2H), 2.20 (s, 3H), 2.08-2.06 (t, J=6.6, 2H), 1.87 (s, 6H), 1.45 (s, 6H).

Intermediate N (1-(8-Bromo-2-quinolinyl) ethenone) was prepared as follows: Under an inert atmosphere, a 250 mL Schlenk flask was loaded with dry and deoxygenated THF (15 mL) followed by addition of ethylvinylether (4.6 mL, 47 mmol, 4.0 equiv.). The solution was cooled to −40° C. and n-BuLi (2.5 M in hexanes, 9.5 mL, 23.7 mmol, 2 equiv.) was added dropwise. After complete addition, the reaction was warmed to room temperature over 15 min and stirred at room temperature for 1 h. The Schlenk flask was then cooled to −40° C. and dry ZnBr₂ (0.5 M in THF, 47 mL, 23.7 mmol, 2.0 equiv.) was added. The reaction mixture was warmed to room temperature over 15 min and stirred at room temperature for 15 additional min. Pd₂dba₃ (123 mg, 0.107 mmol, 0.9 mol %) and triphenylphosphine (118 mg, 0.450 mmol, 3.8 mol %) were dissolved in dry and deoxygenated THF (15 mL) and added to the reaction mixture at room temperature for 15 min. A solution of 2,8-dibromoquinoline (3.40 g, 11.9 mmol, 1.0 equiv.) in dry and deoxygenated THF (15 mL) was then added at room temperature and stirred for 16 h. The reaction mixture was heated to 65° C. for 4 hr. Then, aqueous HCL (1M, 25 mL) was added, the reaction was stirred for another 4 hr at 65° C. The reaction was cooled to room temperature and quenched by addition of saturated aqueous NaHCO₃ (5 mL), water (15 mL), and tertbutylmethylether (TBME) (2×25 mL). The organic layer was collected, and the aqueous layer extracted with TBME (2×25 mL). The combined organic layers were dried over MgSO₄ and concentrated under reduced pressure. The brown residue was purified via column chromatography (SiO₂, eluent: toluene, the third spot on the TLC plate corresponds to the product) to yield the Intermediate N compound as a tan-yellow solid (1.34 g, 45% yield). ¹H NMR (400 mHz, CDCl₃): 8.24 (d, 1H), 8.14 (d, 1H), 8.09 (d, 1H), 7.81 (d, 1H), 7.47 (t, 1H), 2.94 (s, 3H). The chemical structure of Intermediate N is shown below:

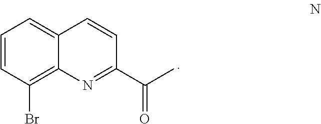

Alternatively, the Intermediate N compound can be synthesized through the use of Intermediate O and Intermediate P compounds as described below.

Intermediate O (8-Bromo-2-quinolinecarboxaldehyde) was prepared as follows. A solution of 8-bromo-2-methylquinoline (0.16 mol) was added to a hot solution of selenium dioxide (0.9 mol) in dioxane and the reaction mixture was heated under reflux for 5 h, then filtered while hot. The solvent was removed in vacuo and purified via column chromatography (dichloromethane) to yield the Intermediate O compound as a pale-yellow solid (93% yield). The chemical structure of Intermediate O is shown below:

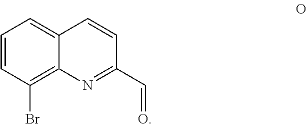 [SMILES: O=Cc1ccc2cccc(Br)c2n1]

Intermediate P (8-Bromo-α-methyl-2-quinolinemethanol) was prepared as follows. A solution of Intermediate O (8-Bromo-2-quinolinecarboxaldehyde) (10.0 mmol, 1.0 equiv.) in THF (100 mL, 0.1 M) was added to an oven dried 250 mL round-bottomed flask under N₂. The mixture was cooled to 0° C. with ice bath, and methyl magnesium bromide (10 mL, 2 M solution in THF, 20.0 mmol, 2.0 equiv.) was added dropwise. The reaction mixture was stirred at 0° C. for 1 h and then warmed to room temperature and stirred for 3 h. The reaction mixture was then cooled to 0° C. and quenched by addition of saturated ammonium chloride solution (40 mL) and brine (100 mL). The aqueous phase was extracted with ethyl acetate (100 mL×2) and the organic phases combined and dried over anhydrous sodium sulfate. The organic fraction was filtered, and the volatiles removed in vacuo. The product was purified via flash column chromatography (silica gel, PE/EtOAc=8:1 to 5:1) to yield the Intermediate P compound as a pale-yellow solid (87%-98% yield). The chemical structure of Intermediate P is shown below:

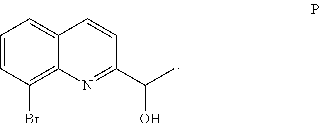

Intermediate N (1-(8-Bromo-2-quinolinyl) ethenone) was alternatively prepared as follows. A solution of Intermediate P (8-Bromo-α-methyl-2-quinolinemethanol) (4.0 mmol, 1.0 equiv.), MnO₂ (2.09 g, 24.0 mmol, 6.0 equiv.) in toluene (40 mL) was added to an oven dried 100 mL round-bottomed flask. The reaction was stirred at room temperature for 20 h. Then, the reaction was filtered and rinsed with ethyl acetate (30 mL×2). The organic phases were combined, and the volatiles removed in vacuo. The product was purified via flash column chromatography (silica gel, PE/EtOAc=8:1) to yield the Intermediate N compound as a pale-yellow solid (90%-95% yield). The chemical structure of Intermediate N is shown below:

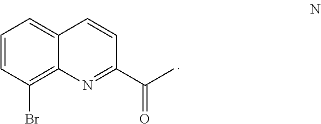

Characterization data for Intermediate compounds N, O, and P can be found, for instance, in Yang, X., Shan, G., Rao, Y., Synthesis of 2-Aminophenols and Heterocycles by Ru-Catalyzed C—H Mono- and Dihydroxylation, Organic Letters 2013, 15 (10), 2334-2337; Sun, H.-R., Zhao, Q., Yang, H., Yang, S., Gou, B.-B., Chen, J., Zhou, L., Chiral Phosphoric-Acid-Catalyzed Cascade Prins Cyclization, Organic Letters 2019, 21 (17), 7143-7148; and Nagy, S., Nifantev, L. E., Neal-Hawkins, K. L., Mihan, S., Catalyst system based on quinoline donors, US Patent Publication 2013/0023634.

The Intermediate Q, R, and S compounds can be synthesized through the use of the Intermediate N compound as described below.

Intermediate Q ((E)-N-(1-8-bromoquinolin-2-yl)ethylidene)-2,4,6-trimethylaniline) was prepared as follows. A solution of 2,4,6-trimethylaniline (1.04 mL, 7.39 mmol), Intermediate N 1-(8-bromoquinlin-2-yl) ethenone (1.54 g, 6.16 mmol) were added to a 25 mL flask and in toluene (10 mL). 4-methylbenzenesulfonic acid (53 mg, 0.308 mmol) was added and the mixture was refluxed under Dean-Stark conditions for 16 h. TLC analysis indicated full conversion of the starting material (SiO₂, eluent: toluene). The reaction solution was cooled to room temperature and filtered over a silica plug eluting with toluene (until no more yellow elutes). The solvent was removed under reduced pressure to yield the Intermediate Q compound as a yellow-orange solid (2.35 g, 99% yield). The chemical structure of Intermediate Q is shown below:

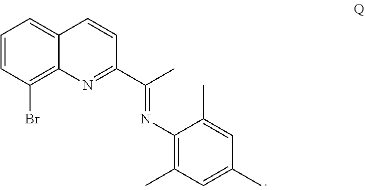 [SMILES: C/C(=N\c1c(C)cc(C)cc1C)c1ccc2cccc(Br)c2n1]

Intermediate R (1-(8-(dicyclohexylphosphino)-2-quinolinyl) ethenone) was prepared as follows. Under inert an atmosphere, a 100 mL flask was charged with diacetoxypalladium (0.056 g, 0.249 mmol, 7.5 mol %), 1,1′-bis(diisopropylphosphino) ferrocene (DiPPF, 0.126 g, 0.227 mmol 9 mol %), sodium tert-butoxide (0.385 g, 4.00 mmol, 1.2 equiv.), and Intermediate N (1.07 g, 4.27 mmol, 1 equiv.), followed by addition of toluene (4 mL, 0.1 M). Dicyclohexylphosphine (0.583 mL, 2.285 mmol, 1.05 equiv.) was added by syringe and stirred for 24 h at 110° C., the reaction was monitored by TCL (SiO₂, toluene). After cooling down to room temperature, degassed sat. aq. NaHCO₃ (10 mL) was added, and extracted. The organic layer was concentrated under reduced pressure affording a black residue. Purification by CC (neutral alumina, toluene/heptane 2:1), 20 g alumina, Fractions 4-11 were collected to yield the Intermediate R compound as a red oil (0.814 g, 55% yield). The chemical structure of Intermediate R is shown below:

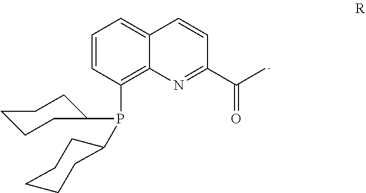 [SMILES: CC(=O)c1ccc2cccc(P(C3CCCCC3)C3CCCCC3)c2n1]

Intermediate S (1-(8-(diphenylphosphino)-2-quinolinyl) ethenone) was prepared as follows. Under inert an atmosphere, a 100 mL flask was charged with diacetoxypalladium (0.056 g, 0.249 mmol, 7.5 mol %), 1,1′-bis(diisopropylphosphino) ferrocene (DiPPF, 0.126 g, 0.277 mmol, 9 mol %), sodium tert-butoxide (0.385 g, 4.00 mmol, 1.2 equiv.), and Intermediate N (1.07 g, 4.27 mmol, 1 equiv.), followed by addition of toluene (4 mL, 0.1 M). Diphenylphosphine (0.583 mL, 3.07 mmol, 1.05 equiv.) was added by syringe and stirred for 24 h at 110° C., the reaction was monitored by TCL: SiO₂, toluene. After cooling down to room temperature, degassed sat. aq. NaHCO₃ (10 mL) was added and extracted. The organic layer was concentrated under reduced pressure affording a black residue. Purification by CC (neutral alumina, toluene/heptane 2:1), 20 g alumina afforded the Intermediate S compound as a brown solid (0.318 g, 45% yield). The chemical structure of Intermediate S is shown below:

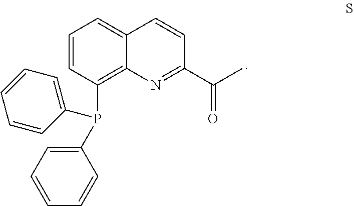 [SMILES: CC(=O)c1ccc2cccc(P(c3ccccc3)c3ccccc3)c2n1]

### SYNTHESIS OF HETEROATOMIC LIGAND COMPOUNDS

The following heteroatomic ligand compounds were synthesized according to the procedures below.

Ligand 1 ((E)-N-(1-8-(diphenylphosphino)-2-yl)ethylidene)-2,4,6-trimethylaniline) was prepared as follows. Under an inert atmosphere, a 100 mL flask was charged with diacetoxypalladium (0.056 g, 0.251 mmol, 7.5 mol %) 1,1′-1,1′ bis(diisopropylphosphino) ferrocene (DiPPF, 0.126 g, 0.301 mmol), sodium tert-butoxide (0.385 g, 4.02 mmol), and Intermediate Q (1.23 g, 3.35 mmol), followed by addition of toluene (34 mL). The diphenylphosphine (0.583 mL, 3.35 mmol) was added by syringe and stirred for 24 h at 110° C. The reaction mixture was filtered over a celite plug and eluted with toluene (2×20 mL) and the filtrate concentrated under reduced pressure. The residue was further purified by column chromatography (neutral alox, heptane to 3:1 heptane/toluene) to yield the desired Ligand 1 product as a yellow solid (1.13 g, 71% yield). The chemical structure of Ligand 1 is presented below in Table 1.

Ligand 2 ((E)-N-(1-8-(diisobutylphosphino)-2-yl)ethylidene)-2,4,6-trimethylaniline) was prepared as follows. Under an inert atmosphere, a 100 mL flask was charged with diacetoxypalladium (7.5 mol %) 1,1′-bis(diisopropylphosphino) ferrocene (DiPPF, 9 mol %), sodium tert-butoxide (1.2 equiv.), and Intermediate Q (1 g, 3.35 mmol, 1.0 equiv.), followed by addition of toluene (18 mL, 0.15 M). The diisobutyl phosphine (1.1 equiv.) was added by syringe and stirred for 16 h at 110° C. The reaction mixture was filtered over celite and concentrated under reduced pressure. The crude product was purified by column chromatography (neutral alumina 20 g; 2:1 hept/tol), to remove the DiPPF impurity, followed by a second column chromatography (silica, 20 g; 20:1 hept/EtOAc), to remove the quinoline impurity, to yield the desired Ligand 2 product (0.447 g, 38% yield and 97% purity). The chemical structure of Ligand 2 is presented below in Table 1.

Ligand 3 ((E)-N-(1-8-(diisopropylphosphino)-2-yl)ethylidene)-2,4,6-trimethylaniline) was prepared as follows. Under an insert atmosphere, a 100 mL flask was charged with diacetoxypalladium (7.5 mol %), 1,1′-bis(diisopropylphosphino) ferrocene (DiPPF, 9 mol %), sodium tert-butoxide (1.2 equiv.), and Intermediate Q (0.863 g, 1.0 equiv.), followed by addition of toluene (16 mL, 0.15 M). The diisopropyl phosphine (1.1 equiv.) was added by syringe and stirred for 16 h at 110° C. The reaction mixture was filtered over celite and concentrated under reduced pressure. The crude product was purified by column chromatography (silica, 20 g; 20:1 hept/EtOAc). The product and impurity have similar R_{f}-values which made the separation challenging. Under these conditions, a fraction was obtained which did not contain the quinoline derived impurity, however the DiPPF coeluted with the desired product (350 mg). To remove the DiPPF impurity, the obtained material was subjected to column chromatography (neutral alumina 20 g, 2:1 hept/tol). This afforded the desired Ligand 3 product (0.249 g, 26% yield and 91% purity). The chemical structure of Ligand 3 is presented below in Table 1.

Ligand 4 ((E)-N-(1-8-(dicyclohexylphosphino)-2-yl)ethylidene)-2,4,6-trimethylaniline) was prepared as follows. Under an inert atmosphere, a 100 mL flask was charged with diacetoxypalladium (5 mol %), 1,1′-bis(diisopropylphosphino) ferrocene (DiPPF, 6 mol %), sodium tert-butoxide (1.2 equiv.), and Intermediate Q (0.549 g, 1 equiv.), followed by addition of toluene (10 mL, 0.13 M). The dicyclohexyl phosphine (1.05 equiv.) was added by syringe and stirred for 16 h at 110° C. Filtration over celite followed by removal of solvent afforded crude product containing 10% of an unidentified side product (no starting material present in the mixture). The crude product was purified by column chromatography (neutral alumina, 2:1 hept/tol). While the DiPPF impurity could be removed with this chromatographic method the quinoline derived impurity remained. It was found that using thoroughly dried silica gel as the stationary phase and heptanes/EtOAc (20:1) as the mobile phase allowed the remaining 10% impurity to be removed. This afforded the desired Ligand 4 product (0.324 g in 49% yield and 99% purity). The chemical structure of Ligand 4 is presented below in Table 1.

Ligand 5 ((E)-N-(1-8-(diphenylphosphino)-2-yl)ethylidene)-2,4-trimethylaniline) was prepared as follows. Under an inert atmosphere, Intermediate S (0.1000 g, 0.281 mmol, 1.0 equiv.) was added to a solution of 2,4-dimethyl aniline (0.04 mL, 0.338 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 5 product as a red solid (220 mg, 82% yield). A cleaner final product was obtained by dissolving the orange solid in anhydrous methanol and crashing out the product in a freezer overnight and collecting the precipitate by filtration. The chemical structure of Ligand 5 is presented below in Table 1.

Ligand 6 ((E)-N-(1-8-(diphenylphosphino)-2-yl)ethylidene)-4-trimethylaniline) was prepared as follows. Under an inert atmosphere, Intermediate S (0.2860 g, 0.805 mmol, 1.0 equiv.) was added to a solution of 4-methyl aniline (0.11 mL, 0.965 mmol, 1.2 eq) in anhydrous toluene, molecular sieves (4 Å, 200 mg) and silica-alumina catalyst support (50 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 6 product as a yellow solid (0.108 g, 86% yield). The chemical structure of Ligand 6 is presented below in Table 1.

Ligand 6-Fe was prepared as follows: Experiment on the feasibility of templated imine condensation to 6-Fe to avoid purification by column chromatography of the labile PIQ-Ligand. A 15 mL vial loaded with p-toluidine (30.2 mg, 0.281 mmol, 1.05 equiv.), Ligand 6 (100 mg, 0.281 mmol, 1.05 equiv.), iron (II) chloride (34.0 mg, 0.268 mmol, 1.0 equiv.) was inertized. Degassed isopropyl alcohol (3.4 mL) was added and the reaction was stirred for 24 h at 50° C. The dark blue mixture was cooled to room temperature, filtered and washed with iPrOAc (3×1 mL). The wet solid was dried under reduced pressure affording the desired Ligand 6-Fe product (Fe4C-1) as a dark blue solid (128 mg, 84% yield). Elemental Analysis: C, 63.08; H, 4.41; Cl, 12.41; Fc, 9.78; N, 4.90; P, 5.42. The chemical structure of Ligand 6-Fe is presented below in Table 1.

Ligand 7 ((E)-N-(1-8-(dicyclohexylphosphino)-2-yl)ethylidene)-2,4-trimethylaniline) was prepared as follows. Under an inert atmosphere, Intermediate R (0.2000 g, 0.544 mmol, 1.0 equiv.) was added to a solution of 2,4-dimethyl aniline (0.08 mL, 0.653 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 7 product as a red solid (0.327 g, 89% yield). The chemical structure of Ligand 7 is presented below in Table 1.

Ligand 8 ((E)-N-(1-8-(dicyclohexylphosphino)-2-yl)ethylidene)-4-trimethylaniline) was prepared as follows. Under an inert atmosphere, Intermediate R (0.2000 g, 0.544 mmol, 1.0 equiv.) was added to a solution of 4-methyl aniline (0.07 mL, 0.652 mmol, 1.2 equiv.) in anhydrous toluene, molecular sieves (4 Å, 400 mg) and silica-alumina catalyst support (100 mg) were added to the reaction mixture. The reaction was stirred at 50° C. for 20 h. After cooling to room temperature, the mixture was filtered and washed with degassed toluene (2×2 mL). The solvent was removed under reduced pressure yielding the desired Ligand 8 product as a yellow solid (0.208 g, 78% yield). The chemical structure of Ligand 8 is presented below in Table 1.

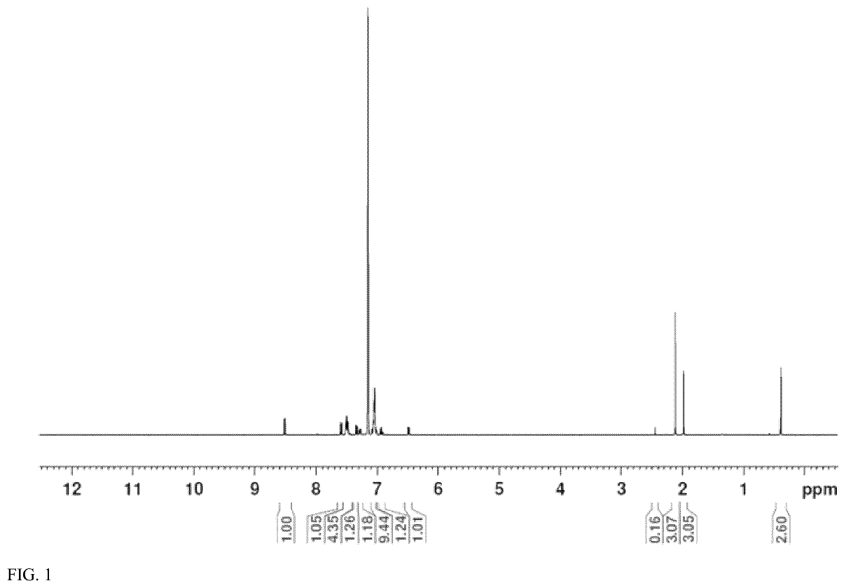

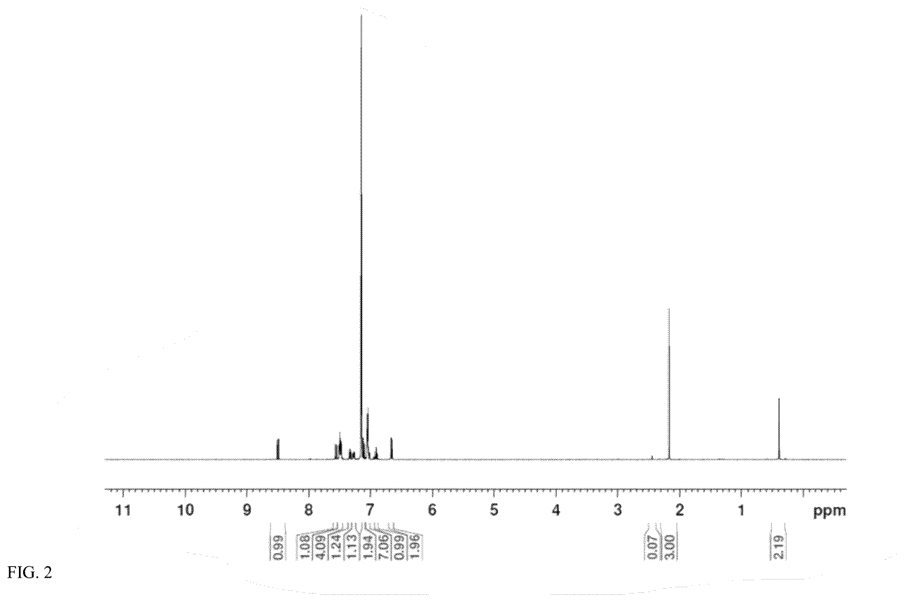

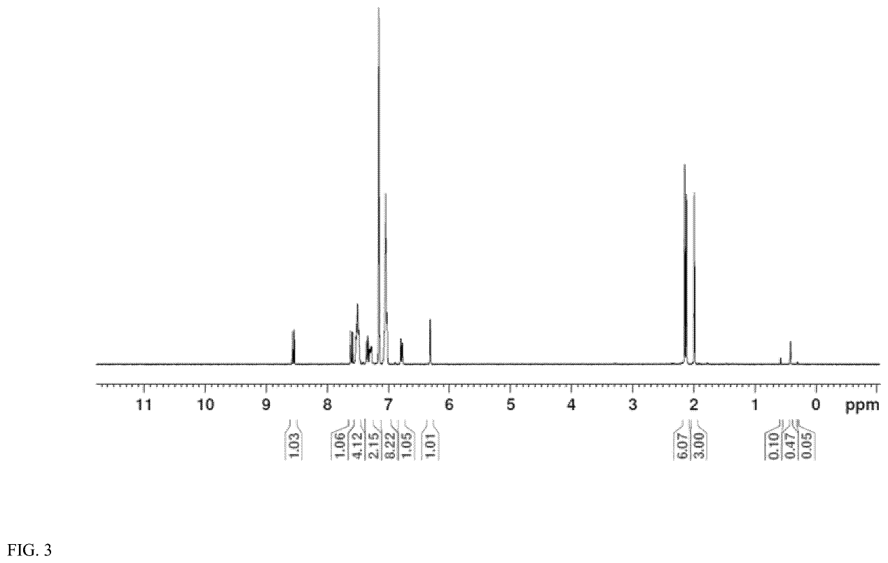

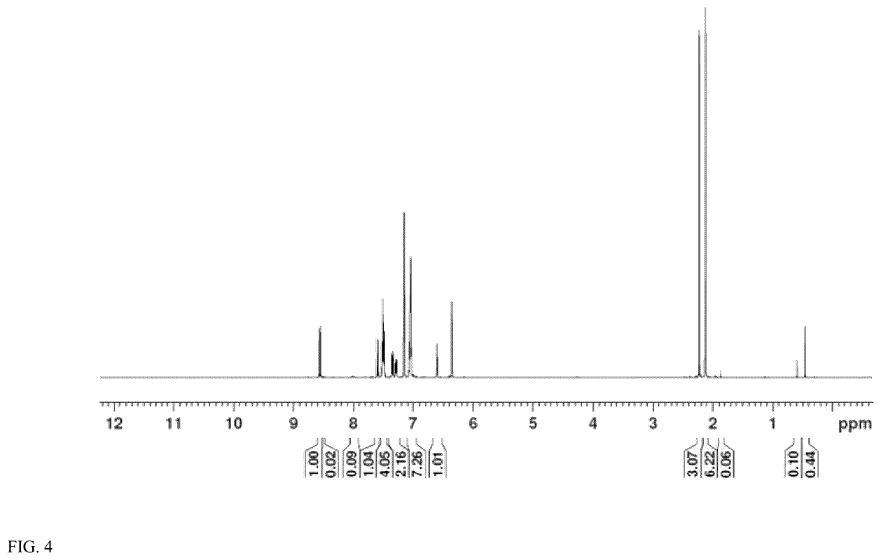

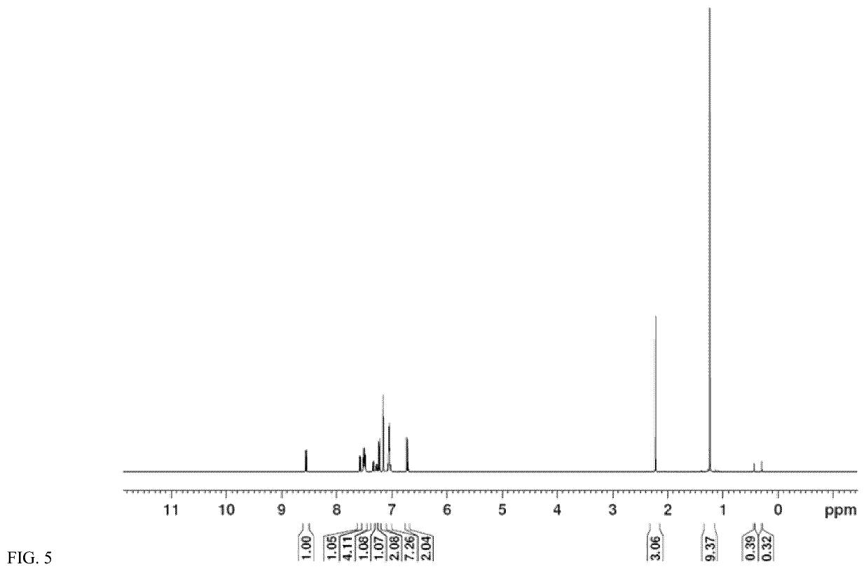

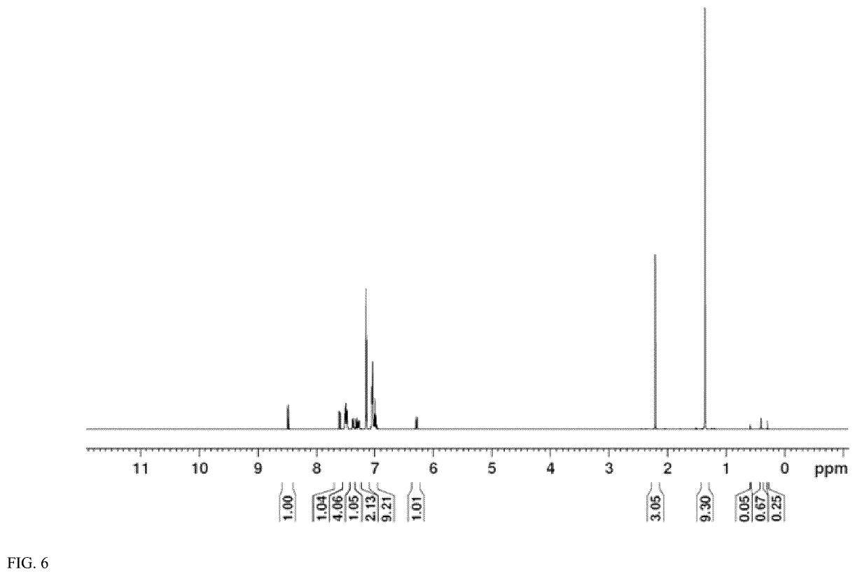

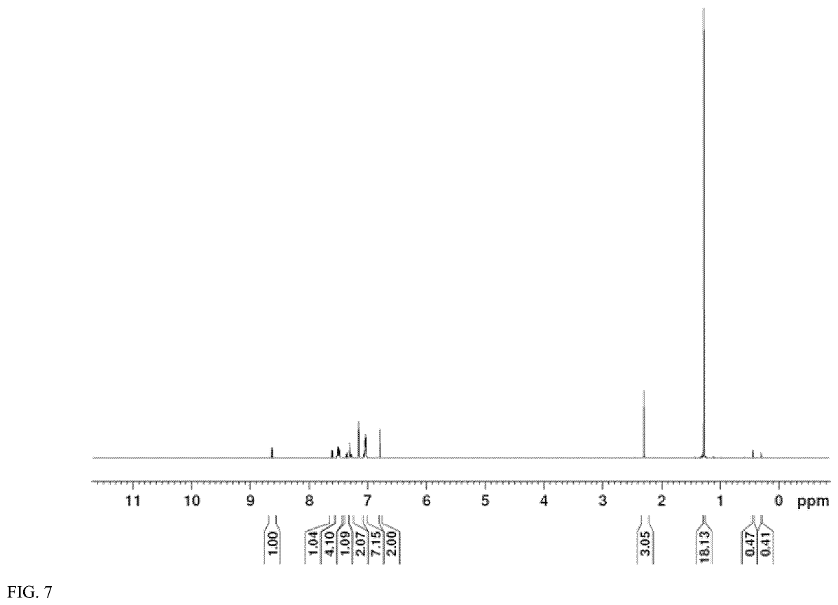

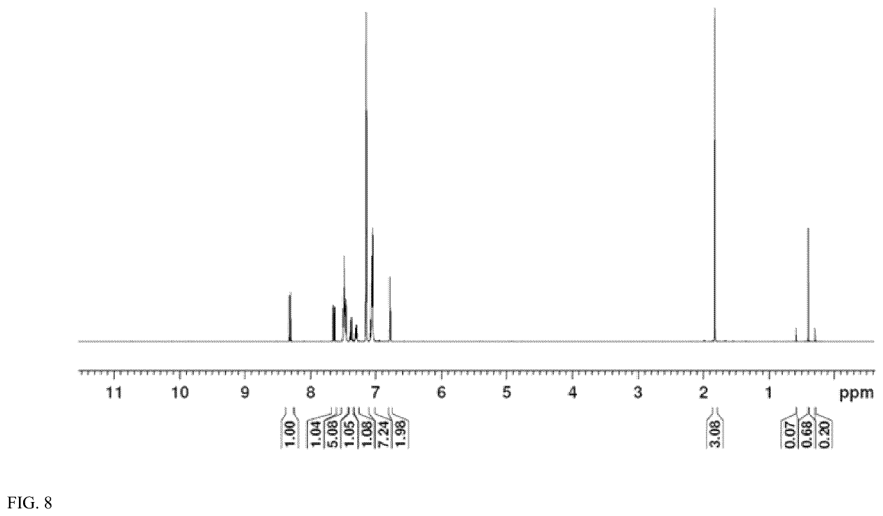

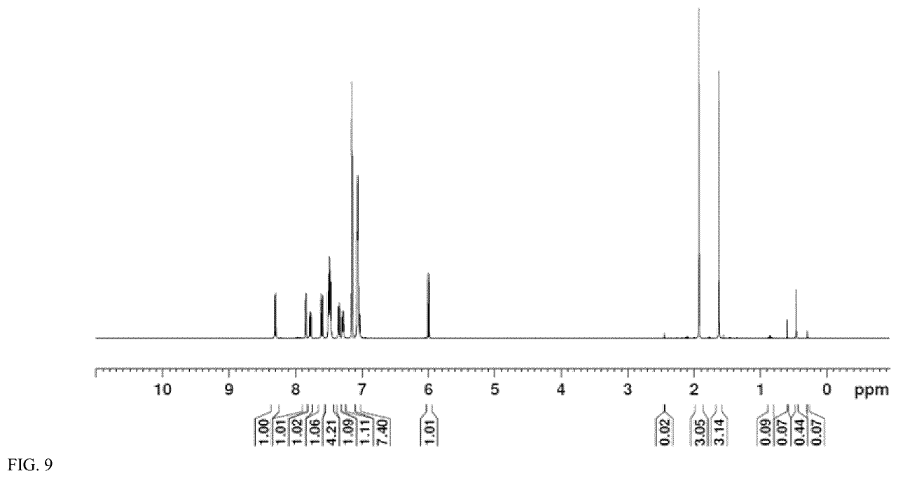

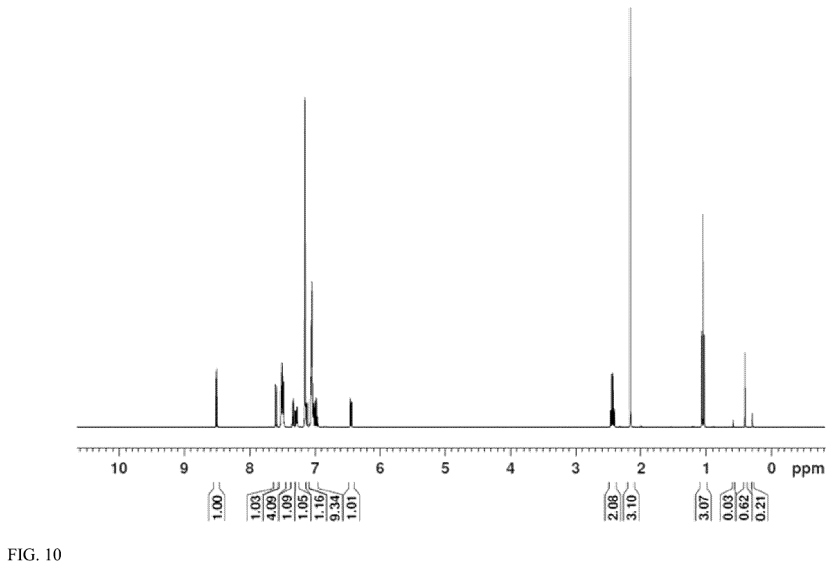

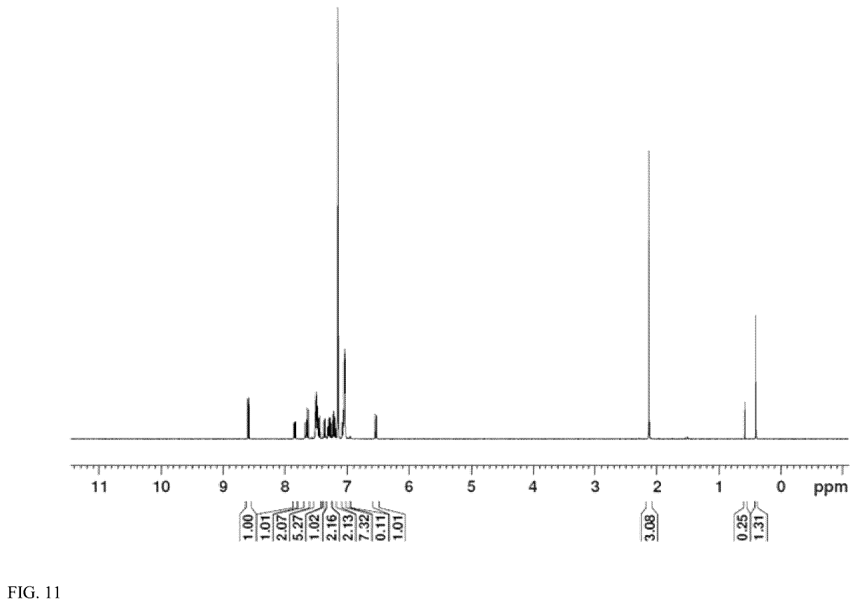

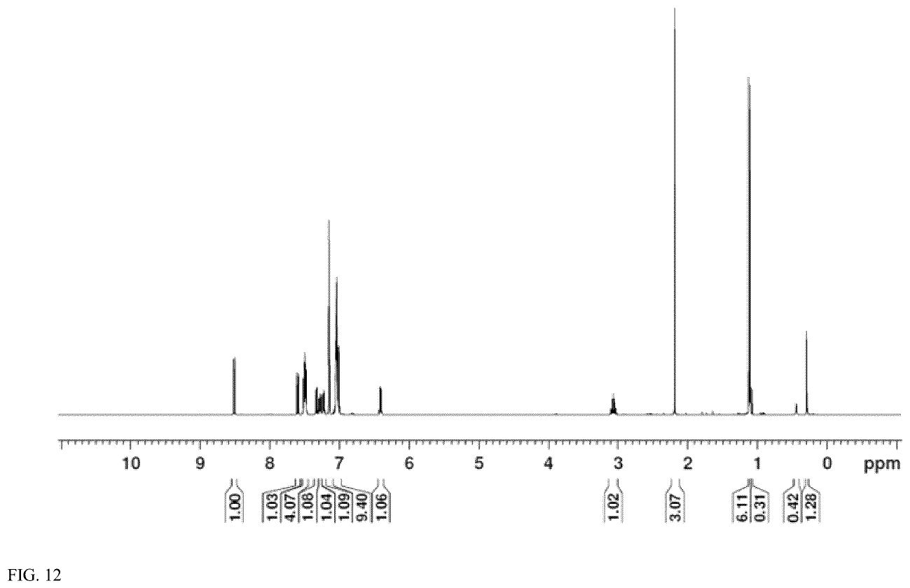

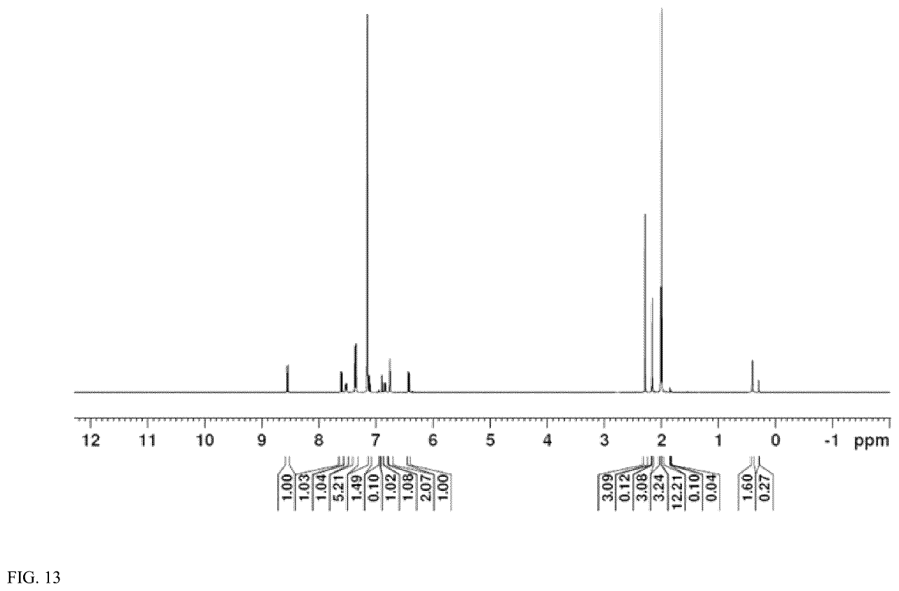

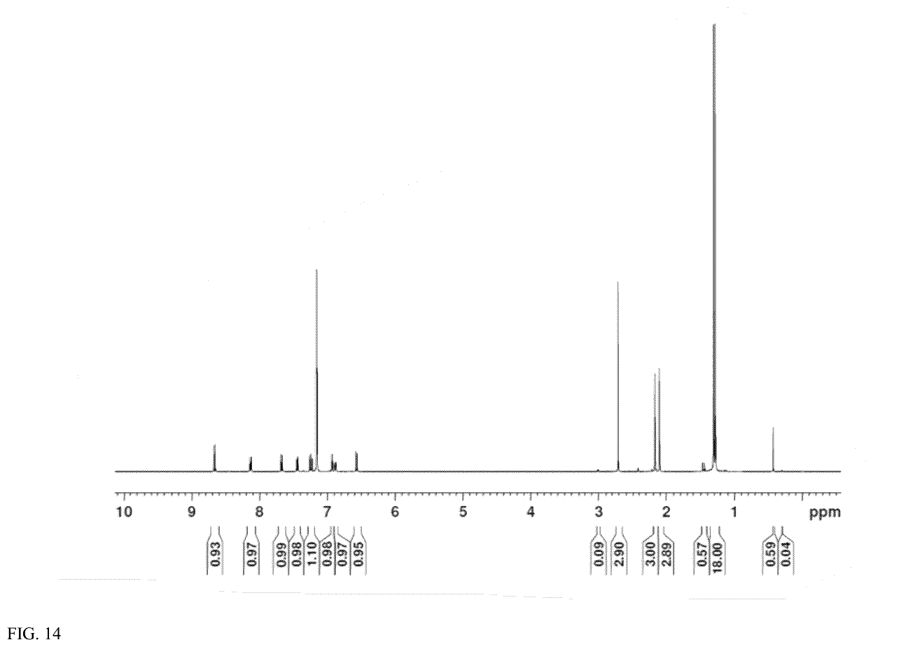

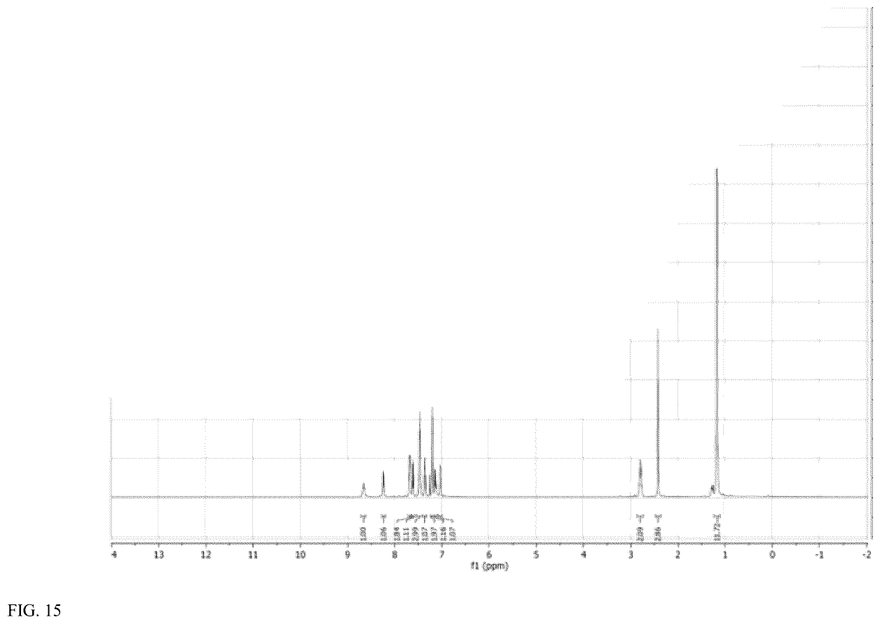

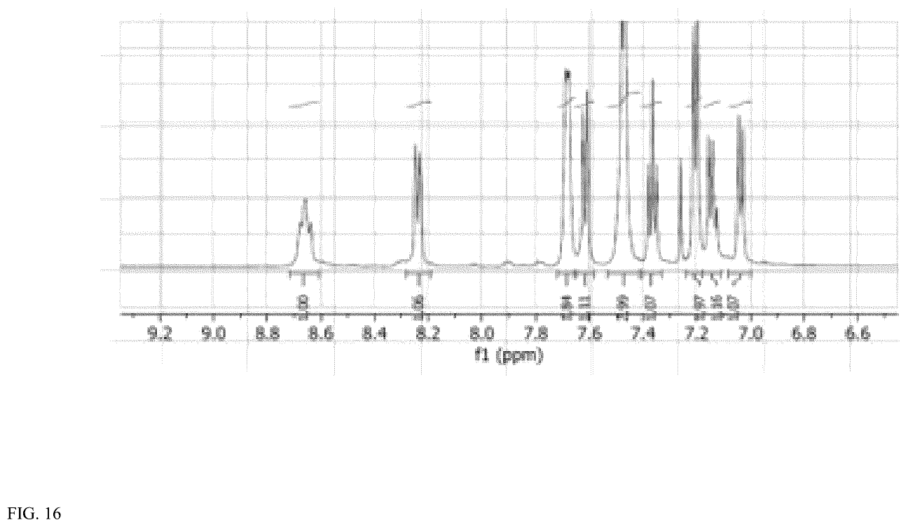

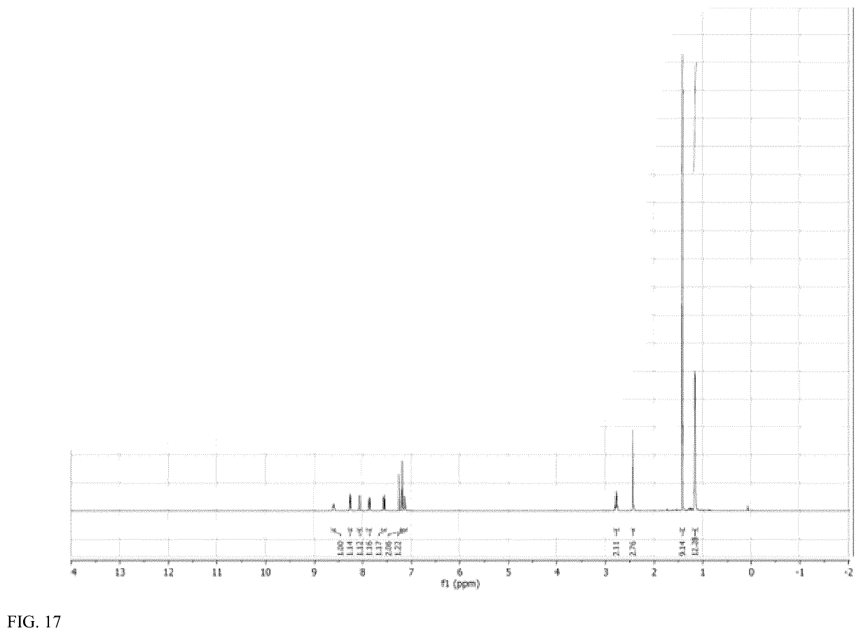

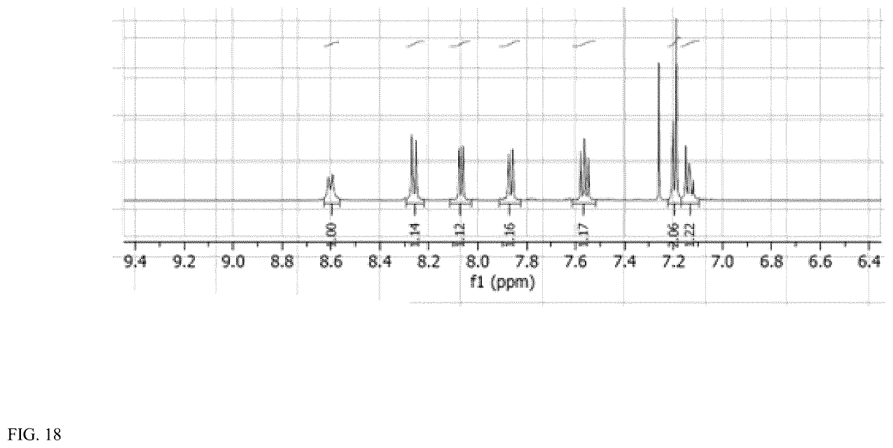

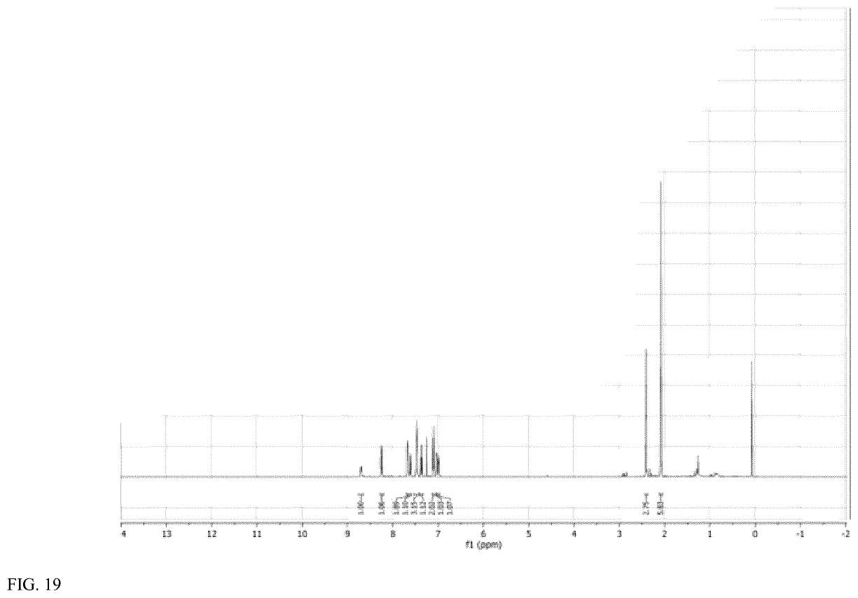

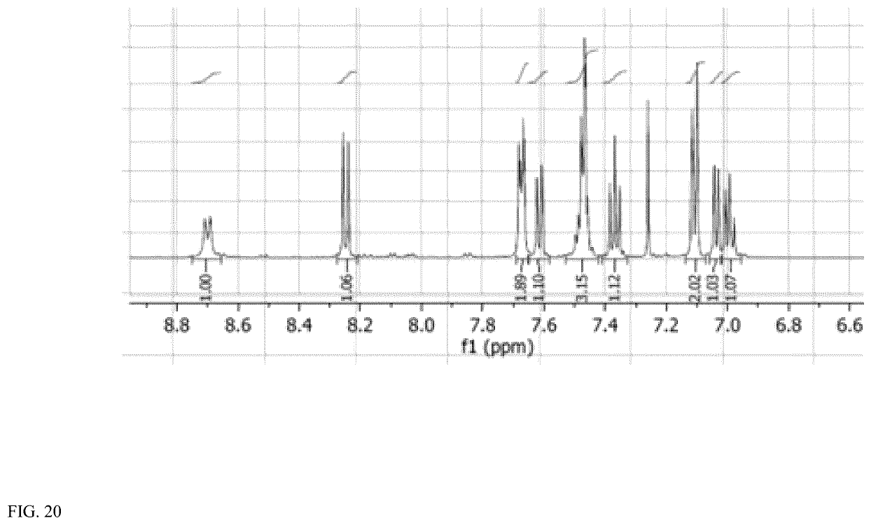

Ligands 9-22 were synthesized using similar techniques to those described hereinabove for Ligands 1-8. The chemical structures for Ligands 9-22 are presented below in Table 1 and ¹H NMR plots confirming the structures for Ligands 9-22 are provided in FIGS. 1-14, respectively. Likewise, Ligands 23-25 were synthesized using similar techniques to those described hereinabove for Ligands 1-8. The chemical structures for Ligands 23-25 are presented below in Table 1 and ¹H NMR plots confirming the structures for Ligands 23-25 are provided in FIGS. 15-16, FIGS. 17-18, and FIGS. 19-20, respectively. Ligand 26 in Table 1 was prepared similarly to Ligands 27-31 described below.

Ligand 27 (1,1-diphenyl-N-(3-(1-(o-tolylimino)ethyl)phenyl)phosphanamine) was prepared as follows. In a glove box, Intermediate G (148 mg, 0.66 mmol) was dissolved in 3 mL of THF (0.2 M) and degassed NEt₃ (0.11 mL, 0.79 mmol, 1.2 equiv.) was added, followed by chlorodiphenylphosphine (0.13 mL, 0.73 mmol, 1.1 equiv.). The reaction mixture was stirred for 16 hr at room temperature. Following filtration, removal of solvent in vacuo yielded the desired Ligand 27 product as a yellow oil (214.0 mg, 80% yield). 1H NMR (500 MHZ, CDCl₃), δ=7.81-7.78 (m, 2H), 7.73-7.70 (m, 1H), 7.60-7.58 (m, 2H), 7.50-7.49 (m, 5H), 7.38 (m, 6H), 7.03-7.01 (d, J=7.99, 1H), 6.84-6.82 (d, J=8.52, 1H), 6.61-6.57 (m, 2H), 5.28-5.26 (d, J=9.03 1H), 1.85 (s, 3H), 1.55 (s, 3H); ³¹P NMR (202 MHZ, CDCl₃), δ 59.9, 27.2. The chemical structure of Ligand 27 is presented below in Table 1.

Ligand 28 (N-(3-(1-((2,4-dimethylphenyl)imino)ethyl)phenyl)-1,1-diphenylphosphanamine was prepared as follows. In a glove box, Intermediate E (148 mg, 0.66 mmol) was dissolved in 3 mL of THF (0.2 M) and degassed NEt₃ (0.11 mL, 0.79 mmol, 1.2 equiv.) was added, followed by chlorodiphenylphosphine (0.13 mL, 0.73 mmol, 1.1 equiv.). The reaction mixture was stirred for 16 hr at room temperature. Following filtration, removal of solvent in vacuo yielded the desired Ligand 28 product as a yellow oil (0.214 g, 80% yield). ¹H NMR (500 MHz, CDCl₃), δ=7.70-7.68 (d, J=6.91, 1H), 7.60-7.57 (t, J=8.24, 2H), 7.51-7.48 (m, 5H), 7.39-7.37 (m, 6H), 7.09-7.07 (d, J=8.46, 1H), 7.01 (s, 1H), 6.97-6.95 (d, J=7.62, 1H), 6.54-6.52 (d, J=8.04, 1H), 5.26-5.24 (d, J=8.73, 1H), 2.30 (s, 3H), 2.13 (s, 3H), 2.05 (s, 3H); ³¹P NMR (202 MHZ, CDCl₃), δ 59.9, 27.1. The chemical structure of Ligand 28 is presented below in Table 1.

Ligand 29 (N-(3-(1-(mesitylimino)ethyl)phenyl)-1,1-diphenyl phosphanamine) was prepared as follows. In a glove box, Intermediate D (250 mg, 0.987 mmol) was dissolved in 5 mL of THF (0.2 M) and degassed NEt₃ (0.16 mL, 1.18 mmol, 1.2 equiv.) was added, followed by chlorodiphenylphosphine (0.19 mL, 1.09 mmol, 1.1 equiv.). The reaction mixture was stirred for 16 hr at room temperature. Following filtration, removal of solvent in vacuo yielded the desired Ligand 29 product as an orange yellow oil (0.368 g, 85% yield). ¹H NMR (500 MHZ, CDCl₃), δ=7.75-7.73 (d, J=7.6, 1H), 7.61-7.58 (t, J=8.14, 1H), 7.50 (m, 5H), 7.38-7.37 (m, 6H), 7.11-7.09 (d, J=8.14, 1H), 6.85 (s, 2H), 5.28-5.26 (d, J=7.25, 1H), 2.27 (s, 3H), 2.02 (s, 3H), 1.97 (s, 6H); ³¹P NMR (202 MHZ, CDCl₃), δ 60.6, 27.1. The chemical structure of Ligand 29 is presented below in Table 1.

Ligand 30 (N-(3-(1-((2,6-dimethylphenyl)imino)ethyl)phenyl)-1,1 diphenylphosphanamine) was prepared as follows. In a glove box, Intermediate F (250 mg, 1.05 mmol) was dissolved in 5.3 mL of THF and degassed NEt₃ (0.17 mL, 1.25 mmol, 1.2 equiv.) was added followed by chlorodiphenylphosphine (0.21 mL, 1.15 mmol, 1.1 equiv.). The reaction mixture was stirred for 16 hr at room temperature. Following filtration, removal of solvent in vacuo yielded the desired Ligand 30 product as a yellow oil (0.400 g, 44.4% yield). ¹H NMR (500 MHZ, CDCl₃), δ 7.78-7.76 (d, J=5.5, 1H), 7.62-7.59 (t, J=7.9, 1H), 7.50-7.48 (m, 6H), 7.39-7.36 (m, 4H), 7.04-7.02 (d, J=7.5, 2H), 7.00-6.98 (d, J=7.5, 1H), 6.92-6.89 (t, J=7.4, 1H), 5.28-5.26 (d, J=7.5, 1H), 2.03 (s, 3H), 2.01 (s, 3H), 1.93 (s, 1H); ³¹P NMR (202 MHZ, CDCl₃), δ 60.63, 27.14. The chemical structure of Ligand 30 is presented below in Table 1.

Ligand 31 (1,1-diphenyl-N-(3-(1-(p-tolylimino)ethyl)phenyl)phosphanamine) was prepared as follows. In a glove box, Intermediate H (250 mg, 1.11 mmol) was dissolved in 5.6 mL of THF and degassed NEt₃ (0.19 mL, 1.33 mmol, 1.2 equiv.) was added, followed by chlorodiphenylphosphine (0.22 mL, 1.22 mmol, 1.1 equiv.). The reaction mixture was stirred for 16 hr at room temperature. Following filtration, removal of solvent in vacuo yielded the desired Ligand 31 product as a yellow oil (0.349 g, 64% yield). ¹H NMR (500 MHz, CDCl₃), δ=7.63-7.61 (d, J=8.55, 1H), 7.59-7.56 (t, J=7.48, 1H), 7.49-7.48 (m, 5H), 7.38-7.37 (m, 6H), 7.15-7.13 (d, J=8.0, 2H), 6.70-6.68 (d, J=8.2, 2H), 6.63-6.61 (d, J=8.2, 1H), 5.29 (brs, 1H), 2.34 (s, 3H), 2.19 (s, 3H); ³¹P NMR (202 MHz, CDCl₃), δ 59.82, 27.24. The chemical structure of Ligand 31 is presented below in Table 1.

Ligand 34 ((E)-5-((3,5-dimethylphenyl)thio)-N-mesityl-3,3-dimethyl-2,3-dihydroacridin-4 (1H)-imine) was prepared as follows. In a glove box, Intermediate M ((E)-5-bromo-N-mesityl-3,3-dimethyl-2,3-dihydroacridin-4 (1H)-imine) (100 mg, 0.25 mmol, 1 equiv.), Pd(OAc)₂ (2.9 mg, 5 mol %), DiPPF (8.5 mg, 6 mol %), NaOtBu (29.4 mg, 0.3 mmol, 1.2 equiv.) and toluene (2.5 mL) were combined and stirred for 1 h at room temperature. Then, 3,5-dimethylthiophenyl (35.2 mg, 0.25 mmol, 1 equiv.) was added to the reaction solution and stirred for 24 h at 120° C. The solvent was removed in vacuo and purified via flash chromatography (hexanes:EtOAc 5:1) to yield the desired Ligand 34 product as a deep red solid (0.096 g, 81% yield). ¹H NMR (500 MHZ, CDCl₃), δ=7.88 (s, 1H), 7.32-7.30 (d, J=8.1, 1H), 7.16-7.13 (t, J=7.6, 1H), 7.12 (s, 2H), 7.05 (s, 1H), 6.75 (s, 2H), 6.70-6.69 (dd, J=7.5, 1.2, 1H), 3.12-3.09 (t, J=6.7, 2H), 2.36 (s, 6H), 2.23 (s, 3H), 2.08-2.05 (t, J=6.7, 2H), 1.90 (s, 6H), 1.46 (s, 6H). The chemical structure of Ligand 34 is shown below:

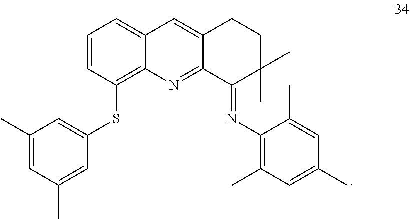

TABLE 1

| | |
|---|---|
| 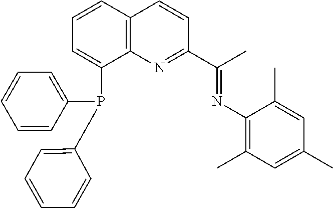 [SMILES: C/C(=N\c1c(C)cc(C)cc1C)c1ccc2cccc(P(c3ccccc3)c3ccccc3)c2n1] | 1 |
| 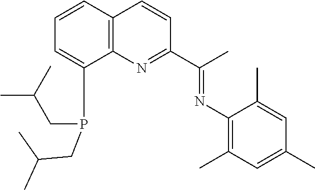 [SMILES: C/C(=N\c1c(C)cc(C)cc1C)c1ccc2cccc(P(CC(C)C)CC(C)C)c2n1] | 2 |
| 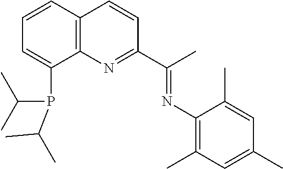 [SMILES: C/C(=N\c1c(C)cc(C)cc1C)c1ccc2cccc(P(C(C)C)C(C)C)c2n1] | 3 |
| 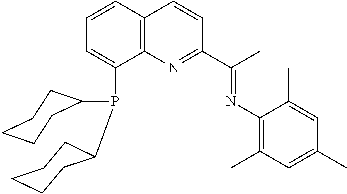 [SMILES: C/C(=N\c1c(C)cc(C)cc1C)c1ccc2cccc(P(C3CCCCC3)C3CCCCC3)c2n1] | 4 |
| 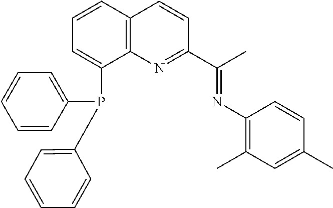 [SMILES: C/C(=N\c1ccc(C)cc1C)c1ccc2cccc(P(c3ccccc3)c3ccccc3)c2n1] | 5 |
| 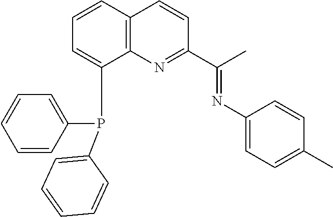 [SMILES: C/C(=N\c1ccc(C)cc1)c1ccc2cccc(P(c3ccccc3)c3ccccc3)c2n1] | 6 |
| 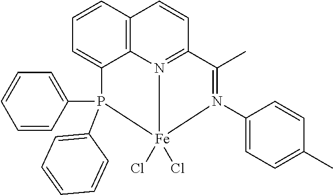 | 6•Fe |
| 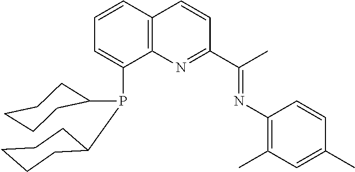 [SMILES: C/C(=N\c1ccc(C)cc1C)c1ccc2cccc(P(C3CCCCC3)C3CCCCC3)c2n1] | 7 |
| 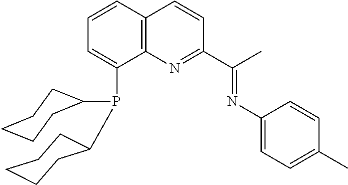 [SMILES: C/C(=N\c1ccc(C)cc1)c1ccc2cccc(P(C3CCCCC3)C3CCCCC3)c2n1] | 8 |
| 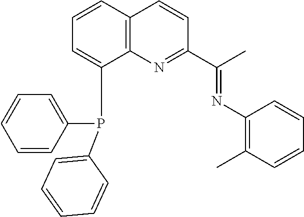 [SMILES: C/C(=N\c1ccccc1C)c1ccc2cccc(P(c3ccccc3)c3ccccc3)c2n1] | 9 |
| 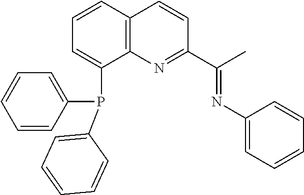 [SMILES: C/C(=N\c1ccccc1)c1ccc2cccc(P(c3ccccc3)c3ccccc3)c2n1] | 10 |
| 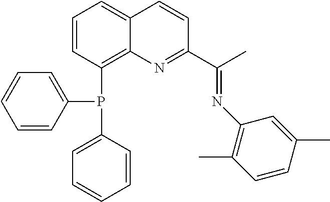 [SMILES: C/C(=N\c1cc(C)ccc1C)c1ccc2cccc(P(c3ccccc3)c3ccccc3)c2n1] | 11 |
| 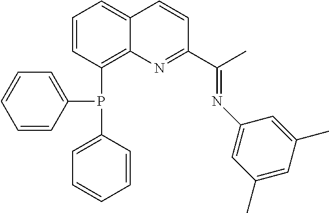 [SMILES: C/C(=N\c1cc(C)cc(C)c1)c1ccc2cccc(P(c3ccccc3)c3ccccc3)c2n1] | 12 |
| 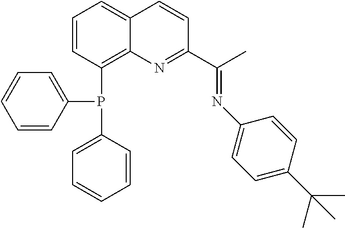 [SMILES: C/C(=N\c1ccc(C(C)(C)C)cc1)c1ccc2cccc(P(c3ccccc3)c3ccccc3)c2n1] | 13 |
| 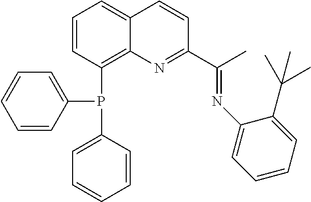 [SMILES: C/C(=N\c1ccccc1C(C)(C)C)c1ccc2cccc(P(c3ccccc3)c3ccccc3)c2n1] | 14 |
| 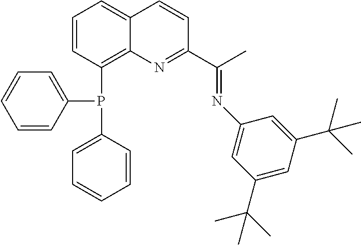 [SMILES: C/C(=N\c1cc(C(C)(C)C)cc(C(C)(C)C)c1)c1ccc2cccc(P(c3ccccc3)c3ccccc3)c2n1] | 15 |
| 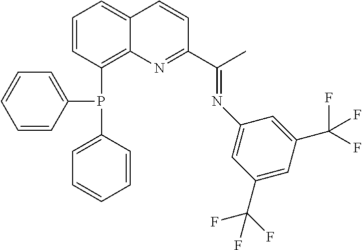 [SMILES: C/C(=N\c1cc(C(F)(F)F)cc(C(F)(F)F)c1)c1ccc2cccc(P(c3ccccc3)c3ccccc3)c2n1] | 16 |
| 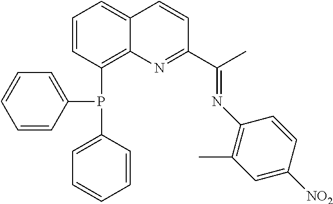 [SMILES: C/C(=N\c1ccc([N+](=O)[O-])cc1C)c1ccc2cccc(P(c3ccccc3)c3ccccc3)c2n1] | 17 |
| 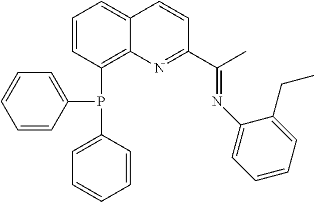 [SMILES: CCc1ccccc1/N=C(\C)c1ccc2cccc(P(c3ccccc3)c3ccccc3)c2n1] | 18 |
| 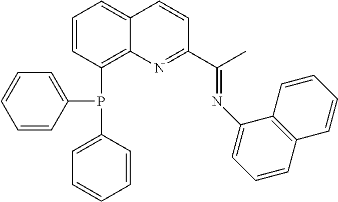 [SMILES: C/C(=N\c1cccc2ccccc12)c1ccc2cccc(P(c3ccccc3)c3ccccc3)c2n1] | 19 |
| 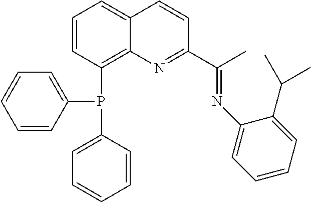 [SMILES: C/C(=N\c1ccccc1C(C)C)c1ccc2cccc(P(c3ccccc3)c3ccccc3)c2n1] | 20 |
| 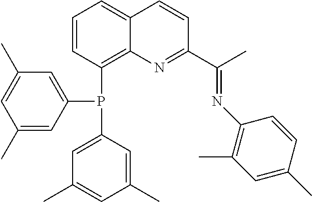 [SMILES: C/C(=N\c1ccc(C)cc1C)c1ccc2cccc(P(c3cc(C)cc(C)c3)c3cc(C)cc(C)c3)c2n1] | 21 |
| 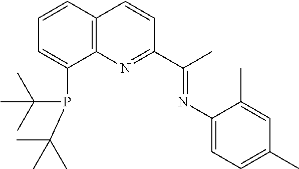 [SMILES: C/C(=N\c1ccc(C)cc1C)c1ccc2cccc(P(C(C)(C)C)C(C)(C)C)c2n1] | 22 |
| 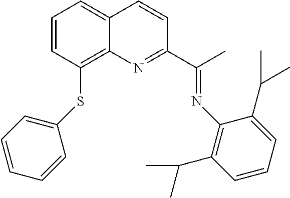 [SMILES: C/C(=N\c1c(C(C)C)cccc1C(C)C)c1ccc2cccc(Sc3ccccc3)c2n1] | 23 |
| 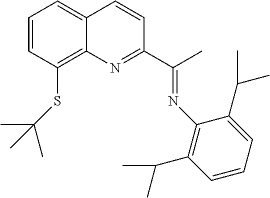 [SMILES: C/C(=N\c1c(C(C)C)cccc1C(C)C)c1ccc2cccc(SC(C)(C)C)c2n1] | 24 |
| 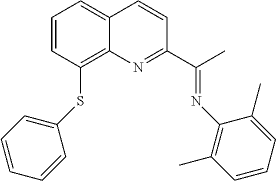 [SMILES: C/C(=N\c1c(C)cccc1C)c1ccc2cccc(Sc3ccccc3)c2n1] | 25 |
| 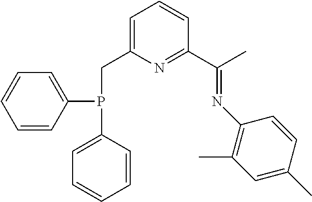 [SMILES: C/C(=N\c1ccc(C)cc1C)c1cccc(CP(c2ccccc2)c2ccccc2)n1] | 26 |
| 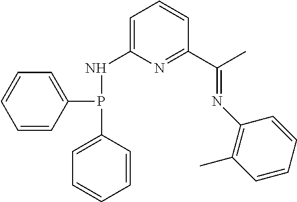 [SMILES: C/C(=N\c1ccccc1C)c1cccc(NP(c2ccccc2)c2ccccc2)n1] | 27 |
| 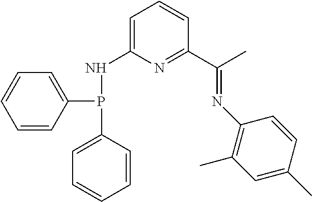 [SMILES: C/C(=N\c1ccc(C)cc1C)c1cccc(NP(c2ccccc2)c2ccccc2)n1] | 28 |
| 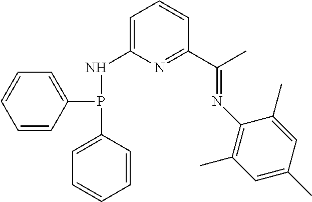 [SMILES: C/C(=N\c1c(C)cc(C)cc1C)c1cccc(NP(c2ccccc2)c2ccccc2)n1] | 29 |
| 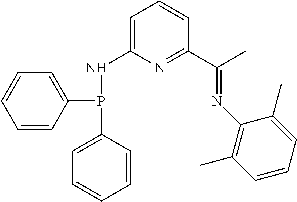 [SMILES: C/C(=N\c1c(C)cccc1C)c1cccc(NP(c2ccccc2)c2ccccc2)n1] | 30 |
| 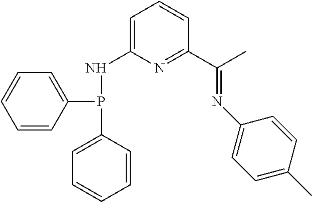 [SMILES: C/C(=N\c1ccc(C)cc1)c1cccc(NP(c2ccccc2)c2ccccc2)n1] | 31 |

### Examples 1-21

The oligomerization experiments of Examples 1-21 were performed as follows. The heteroatomic ligands prepared above, and shown in Table 1, and an excess of a representative soluble iron source (transition metal compound) were combined in a small amount of an aromatic or aliphatic hydrocarbon solvent such as toluene, xylene, or cyclohexane, and added to a sealed NMR tube, which was then affixed to the impeller shaft of a high pressure autoclave, according to the procedure described in Organometallics 2003, 22, 3178 (Small). Cyclohexane solvent (200 mL) and MMAO-3A (modified MAO) were added to the sealed, evacuated autoclave, the reactor was pressurized with ethylene (400-800 psig range), and stirring was begun to break the glass and begin the reaction. Reaction initial (Tᵢₙᵢₜᵢₐₗ) and maximum (Tₘₐₓ) temperatures are indicated in Table 2. Ethylene was fed on demand, and the reactions were terminated by degassing after 15 min. Products were analyzed by gas chromatography, using an internal standard.

The ethylene oligomerization experiments of Examples 1-21 are summarized in Table 2. Yields of volatile products (i.e. C₄) were extrapolated using the Schulz-Flory constant K, with total yields and productivities based on the C₄-C₂₆ products. In some cases, the Schulz-Flory constant is known to drift, typically upwards with increasing carbon number. Therefore, the extrapolation for calculating the K value for C₆/C₄ was based on the rate of change for the three prior fraction measurements. For example, K values for C₁₂/C₁₀, C₁₀/C₈, and C₈/C₆ of 0.52, 0.50, and 0.48, respectively, would give an extrapolated K value of 0.46 for calculating the amount of 1-butene formed.

As shown in Table 2, several examples unexpectedly had molar values of K (nC₁₂/nC₁₀) and K (nC₁₀/nC₈) of 0.5 and below. Further, yields of over 20 g of C₄-C₂₆ products also were achieved, as well as catalyst productivities in the range of 10,000-60,000 g/mmol ligand. The K values for Example 16 (utilizing Ligand 14) were very high (in excess of 0.8-0.9), which typically indicates excessive heavy oligomer or polymer formation. In contrast, the K values for Example 14 (utilizing Ligand 12) were very low (less than 0.2), which typically indicates a preference for dimerization to produce 1-butene. For 1-hexene and 1-octene formation, ordinarily K values in the 0.4-0.6 range are most suitable. Beneficially, several of Examples 1-21 and several different ligands had K values in this general range, such as Examples 6, 9, 11-13, and 17-21.

TABLE 2

| | | Ligand | | | | | K | K | Productivity | |
|---|---|---|---|---|---|---|---|---|---|---|
| | Ligand | MW | MMAO-3A | Metal source | Tᵢₙᵢₜᵢₐₗ | Tₘₐₓ | (nC₁₂/ | (nC₁₀/ | (g/mmol | Yield |
| Example | (mg) | (g/mol) | (Al:Ligand) | (metal:ligand) | (° C.) | (° C.) | nC₁₀) | nC₈) | ligand) | (g C₄-C₂₆) |
| 1 | 1 (0.5) | 470 | 500:1 | Fe(octanoate)₃ | 30 | 62 | 0.786 | 0.767 | 16,290 | 17.3 |
| | | | | (1:1) | | | | | | |
| 2 | 1 (0.5) | 470 | 500:1 | Fe(acac)₂ | 30 | 69 | 0.801 | 0.780 | 12,940 | 13.8 |
| | | | | (4:1) | | | | | | |
| 3 | 2 (0.5) | 432 | 2000:1 | Fe(octanoate)₃ | 30 | 51 | 0.807 | 0.784 | 8,890 | 10.3 |
| | | | | (4:1) | | | | | | |
| 4 | 3 (0.5) | 404 | 2000:1 | Fe(octanoate)₃ | 30 | 44 | 0.851 | 0.825 | 3,690 | 4.6 |
| | | | | (4:1) | | | | | | |
| 5 | 4 (0.48) | 482 | | | 30 | 60 | ND- | | | |
| | | | | | | | waxes | | | |
| 6 | 5 (0.92) | 458 | 1000:1 | Fe(octanoate)₃ | 30 | 90 | 0.514 | 0.489 | 19,100 | 38.2 |
| | | | | (4:1) | | | | | | |
| 7 | 6 (0.89) | 444 | 1000:1 | Fe(octanoate)₃ | 30 | 90 | 0.337 | 0.309 | 26,920 | 53.0 |
| | | | | (4:1) | | | | | | |
| 8 | 5 (0.45) | 458 | 1000:1 | Fe(octanoate)₃ | 75 | 112 | 0.460 | | 30,930 | 30.4 |
| | | | | (4:1) | | | | | | |
| 9 | 7 (0.94) | 470 | 1000:1 | Fe(octanoate)₃ | 30 | 55 | 0.449 | 0.421 | 6,620 | 13.2 |
| | | | | (4:1) | | | | | | |
| 10 | 8 (0.92) | 456 | 1000:1 | Fe(octanoate)₃ | 30 | 82 | 0.194 | 0.157 | 24,350 | 49.1 |
| | | | | (4:1) | | | | | | |
| 11 | 9 (0.43) | 444 | 1000:1 | Fe(octanoate)₃ | 30 | 101 | 0.493 | 0.465 | 26,780 | 26.8 |
| | | | | (4:1) | | | | | | |
| 12 | 9 (0.43) | 444 | 1000:1 | Fe(octanoate)₃ | 75 | 100 | | 0.446 | 30,560 | 30.5 |
| | | | | (4:1) | | | | | | |
| 13 | 11 (0.46) | 458 | 1000:1 | Fe(octanoate)₃ | 75 | 95 | 0.454 | 0.438 | 10,140 | 9.2 |
| | | | | (4:1) | | | | | | |
| 14 | 12 (0.46) | 458 | 1000:1 | Fe(octanoate)₃ | 75 | 125 | 0.199 | 0.171 | 55,240 | 55.3 |
| | | | | (4:1) | | | | | | |
| 15 | 13 (0.49) | 486 | 1000:1 | Fe(octanoate)₃ | 75 | 79 | 0.296 | 0.257 | 2,880 | 2.7 |
| | | | | (4:1) | | | | | | |
| 16 | 14 (0.49) | 486 | 1000:1 | Fe(octanoate)₃ | 75 | 102 | 0.912 | 0.880 | 1,120 | 0.4 |
| | | | | (4:1) | | | | | | |
| 17 | 17 (0.49) | 490 | 1000:1 | Fe(octanoate)₃ | 75 | 103 | 0.605 | 0.559 | 10,850 | 8.1 |
| | | | | (4:1) | | | | | | |
| 18 | 18 (0.46) | 459 | 1000:1 | Fe(octanoate)₃ | 75 | 78 | 0.590 | 0.546 | 3,550 | 2.8 |
| | | | | (4:1) | | | | | | |
| 19 | 27 (0.41) | 409 | 1000:1 | Fe(octanoate)₃ | 75 | 80 | | 0.493 | 6,680 | 6.7 |
| | | | | (4:1) | | | | | | |
| 20 | 28 (0.42) | 423 | 1000:1 | Fe(octanoate)₃ | 75 | 81 | | 0.533 | 13,190 | 13.1 |
| | | | | (4:1) | | | | | | |
| 21 | 29 (0.44) | 438 | 1000:1 | Fe(octanoate)₃ | 75 | 77 | | 0.465 | 11,330 | 11.3 |
| | | | | (4:1) | | | | | | |

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A heteroatomic ligand transition metal compound complex having the following formula:

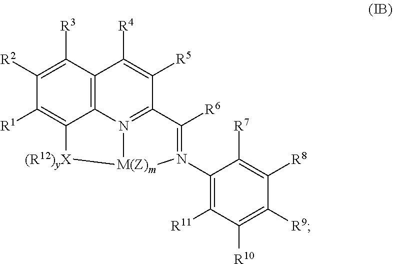

wherein:
X is P or S;
y is equal to 1 when X is S, and y is equal to 2 when X is P;
M is Fe, Co, or Cr;
m is an oxidation state of M;
each Z independently is H, a halogen, a C₁-C₁₈ hydrocarbyl group, or a C₁-C₁₈ halogenated hydrocarbyl group;
R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and
each R¹² independently is a C₁-C₁₈ hydrocarbyl group or a C₁-C₁₈ halogenated hydrocarbyl group.

Aspect 2. The complex defined in aspect 1, wherein M is Fe.

Aspect 3. The complex defined in aspect 1 or 2, wherein each Z is Cl.

Aspect 4. A heteroatomic ligand (compound) having the following formula:

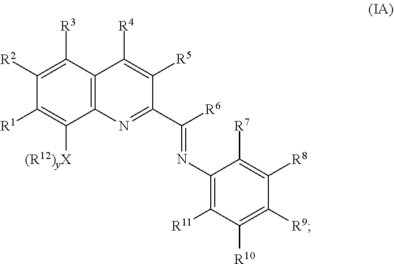

wherein:
X is P or S;
y is equal to 1 when X is S, and y is equal to 2 when X is P;
R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and
each R¹² independently is a C₁-C₁₈ hydrocarbyl group or a C₁-C₁₈ halogenated hydrocarbyl group.

Aspect 5. The complex or ligand defined in any one of aspects 1-4, wherein X is P and y equals 2.

Aspect 6. The complex or ligand defined in any one of aspects 1-4, wherein X is S and y equals 1.

Aspect 7. The complex or ligand defined in any one of aspects 1-6, wherein R¹ to R⁶ independently are H or a methyl group.

Aspect 8. The complex or ligand defined in any one of aspects 1-7, wherein R⁵ and R⁶ are joined to form a ring or ring system.

Aspect 9. The complex or ligand defined in any one of aspects 1-8, wherein R⁷ to R¹¹ independently are H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a nitro group, a pentafluorophenyl group, or a trifluoromethyl group (CF₃).

Aspect 10. The complex or ligand defined in any one of aspects 1-8, wherein R⁷ to R¹¹ independently are H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group, a pentafluorophenyl group, or a trifluoromethyl group (CF₃).

Aspect 11. The complex or ligand defined in any one of aspects 1-10, wherein each R¹² independently is a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group, a naphthyl group, a benzyl group, a pentafluorophenyl group, or a trifluoromethyl group (CF₃).

Aspect 12. The complex or ligand defined in any one of aspects 1-10, wherein each R¹² independently is a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

Aspect 13. A heteroatomic ligand transition metal compound complex having the following formula:

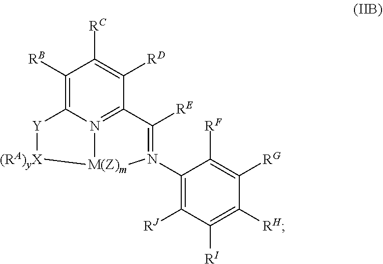

wherein:
X is P or S;
y is equal to 1 when X is S, and y is equal to 2 when X is P; Y is O, NH, or CH₂;
M is Fe, Co, or Cr;
m is an oxidation state of M;
each Z independently is H, a halogen, a C₁-C₁₈ hydrocarbyl group, or a C₁-C₁₈ halogenated hydrocarbyl group;
R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and
each R^{A} independently is a C₁-C₁₈ hydrocarbyl group or a C₁-C₁₈ halogenated hydrocarbyl group.

Aspect 14. The complex defined in aspect 13, wherein M is Fe.

Aspect 15. The complex defined in aspect 13 or 14, wherein each Z is Cl.

Aspect 16. A heteroatomic ligand (compound) having the following formula:

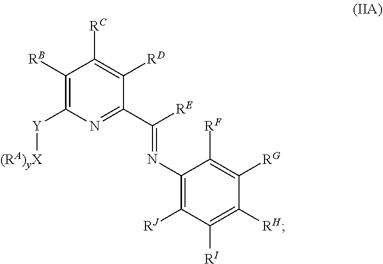

wherein:
X is P or S;
y is equal to 1 when X is S, and y is equal to 2 when X is P; Y is O, NH, or CH₂;
R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and
each R^{A} independently is a C₁-C₁₈ hydrocarbyl group or a C₁-C₁₈ halogenated hydrocarbyl group.

Aspect 17. The complex or ligand defined in any one of aspects 13-16, wherein X is P and y equals 2.

Aspect 18. The complex or ligand defined in any one of aspects 13-16, wherein X is S and y equals 1.

Aspect 19. The complex or ligand defined in any one of aspects 13-18, wherein Y is O.

Aspect 20. The complex or ligand defined in any one of aspects 13-18, wherein Y is NH.

Aspect 21. The complex or ligand defined in any one of aspects 13-18, wherein Y is CH₂.

Aspect 22. The complex or ligand defined in any one of aspects 13-21, wherein R^{B} to R^{E} independently are H or a methyl group.

Aspect 23. The complex or ligand defined in any one of aspects 13-22, wherein R^{D} and R^{E} are joined to form a ring or ring system.

Aspect 24. The complex or ligand defined in any one of aspects 13-23, wherein R^{F} to R^{J} independently are H, Cl, F, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a nitro group, a pentafluorophenyl group, or a trifluoromethyl group (CF₃).

Aspect 25. The complex or ligand defined in any one of aspects 13-23, wherein R^{F} to R^{J} independently are H, Cl, F, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a nitro group, a pentafluorophenyl group, or a trifluoromethyl group (CF₃).

Aspect 26. The complex or ligand defined in any one of aspects 13-25, wherein each R^{A} independently is a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group, a naphthyl group, a benzyl group, a pentafluorophenyl group, or a trifluoromethyl group (CF₃).

Aspect 27. The complex or ligand defined in any one of aspects 13-25, wherein each R^{A} independently is a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, a phenyl group, a methyl-substituted phenyl group, a pentafluorophenyl group, or a trifluoromethyl group (CF₃).

Aspect 28. A catalyst composition comprising (a) the heteroatomic ligand transition metal compound complex defined in any one of aspects 1-3, 5-15, and 17-27; and (b) an organoaluminum compound.

Aspect 29. A catalyst composition comprising (A) the heteroatomic ligand defined in any one of aspects 4-12 and 16-27; (B) a transition metal compound; and (C) an organoaluminum compound.

Aspect 30. The composition defined in aspect 28 or 29, wherein the catalyst composition further comprises a hydrocarbon diluent (or solvent).

Aspect 31. The composition defined in aspect 30, wherein the hydrocarbon diluent (or solvent) comprises a saturated aliphatic hydrocarbon, an aromatic hydrocarbon, or any combination thereof.

Aspect 32. The composition defined in aspect 30, wherein the hydrocarbon diluent (or solvent) comprises a saturated aliphatic hydrocarbon, e.g., propane, butane, pentane, hexane, heptane, octane, cyclohexane, methyl cyclohexane, or combinations thereof.

Aspect 33. The composition defined in aspect 30, wherein the hydrocarbon diluent (or solvent) comprises cyclohexane.

Aspect 34. The composition defined in aspect 30, wherein the hydrocarbon diluent (or solvent) comprises an aromatic hydrocarbon, e.g., benzene, toluene, xylene, cumene, ethylbenzene, or combinations thereof.

Aspect 35. The composition defined in any one of aspects 29-34, wherein the transition metal compound has the formula M(X¹)ₚ, wherein M is Fe, Co, or Cr; p is an oxidation state of M; and each X¹ independently is a monoanionic ligand.

Aspect 36. The composition defined in aspect 35, wherein each X¹ independently is a halogen, a carboxylate, a β-diketonate, a hydrocarboxide, a nitrate, or a chlorate.

Aspect 37. The composition defined in aspect 35, wherein each X¹ independently is acetate, a propionate, a butyrate, a pentanoate, a hexanoate, a heptanoate, an octanoate, a nonanoate, a decanoate, an undecanoate, a dodecanoate, or acetylacetonate.

Aspect 38. The composition defined in any one of aspects 28-37, wherein the organoaluminum compound comprises an aluminoxane, an alkylaluminum compound, or any combination thereof.

Aspect 39. The composition defined in any one of aspects 28-38, wherein a molar ratio of Al:transition metal (or Al:ligand) in the catalyst composition is in any range disclosed herein, e.g., from 10:1 to 5,000:1, from 50:1 to 3,000:1, from 50:1 to 3,000:1, from 75:1 to 2,000:1, from 100:1 to 2,000:1, or from 100:1 to 1,000:1.

Aspect 40. An oligomerization process comprising (i) contacting ethylene, the catalyst composition defined in any one of aspects 28-39, an organic reaction medium, and optionally hydrogen, in an oligomerization reactor; (ii) forming an oligomer product in the oligomerization reactor, the oligomer product comprising hexenes and octenes; and (iii) discharging an effluent stream from the oligomerization reactor, the effluent stream comprising unreacted ethylene and the oligomer product.

Aspect 41. The process defined in aspect 40, wherein hydrogen is contacted in the oligomerization reactor.

Aspect 42. The process defined in aspect 41, wherein hydrogen and ethylene are combined and introduced into the reactor separately from the catalyst composition (or catalyst system components).

Aspect 43. The process defined in any one of aspects 40-42, wherein the organic reaction medium comprises a saturated aliphatic hydrocarbon, an aromatic hydrocarbon, or any combination thereof.

Aspect 44. The process defined in any one of aspects 40-43, wherein the organic reaction medium comprises a saturated aliphatic hydrocarbon, e.g., propane, butane, pentane, hexane, heptane, octane, cyclohexane, methyl cyclohexane, or combinations thereof.

Aspect 45. The process defined in any one of aspects 40-44, wherein the organic reaction medium comprises cyclohexane.

Aspect 46. The process defined in any one of aspects 40-45, wherein the organic reaction medium comprises an aromatic hydrocarbon, e.g., benzene, toluene, xylene, cumene, ethylbenzene, or combinations thereof.

Aspect 47. The process defined in any one of aspects 40-46, wherein the catalyst composition is formed and then introduced into the oligomerization reactor.

Aspect 48. The process defined in any one of aspects 40-46, wherein the catalyst composition is formed within the oligomerization reactor.

Aspect 49. The process defined in any one of aspects 40-48, wherein the oligomer product comprises any amount of octenes disclosed herein, e.g., at least 5, 10, 20, 30 or 40 wt. %; a maximum of 99, 95, 92.5, 90, 87.5, or 85 wt. %; or from 5 to 85 wt. %, from 10 to 90 wt. %, from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes, based on the total amount of oligomers in the oligomer product.

Aspect 50. The process defined in any one of aspects 40-49, wherein the oligomer product comprises any amount of hexenes disclosed herein, e.g., at least 10, 15, 20, 25, 30, or 35 wt. %; a maximum of 75, 65, 60, 55, or 50 wt. %; or from 10 to 75 wt. %, from 15 to 65 wt. %, from 20 to 60 wt. %, from 25 to 55 wt. %, or from 30 to 50 wt. % hexenes, based on the total amount of oligomers in the oligomer product.

Aspect 51. The process defined in any one of aspects 40-50, wherein the oligomerization reactor has any ethylene conversion disclosed herein, e.g., at least 20, 30, 35, 40, 45, or 50 wt. %; a maximum of 99, 95, 90, 80, 75, 70, or 65 wt. %; or from 20 to 95 wt. %, from 30 to 90 wt. %, from 40 to 80 wt. %, from 50 to 70 wt. %, or from 55 to 65 wt. % conversion, based on the amount of ethylene entering the reactor and the amount of ethylene in the effluent stream.

What is claimed is:

1. A heteroatomic ligand compound having the following formula: 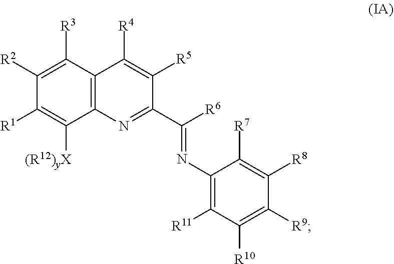
wherein:
X is P or S;
y is equal to 1 when X is S, and y is equal to 2 when X is P;
R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and
each R¹² independently is a C₁-C₁₈ hydrocarbyl group or a C₁-C₁₈ halogenated hydrocarbyl group.

2. The compound of claim 1, wherein:
R¹ to R⁶ independently are H or a methyl group;
R⁷ to R¹¹ independently are H, a methyl group, an ethyl group, a propyl group, a butyl group, a pentyl group, a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, a benzyl group, a nitro group, a pentafluorophenyl group, or a trifluoromethyl group; and
each R¹² independently is a methyl group, an ethyl group, a propyl group, a butyl group, a pentyl group, a hexyl group, a heptyl group, an octyl group, a nonyl group, a decyl group, a cyclobutyl group, a cyclopentyl group, a cyclohexyl group, a cycloheptyl group, an adamantyl group, a phenyl group, a methyl-substituted phenyl group, a naphthyl group, a benzyl group, a pentafluorophenyl group, or a trifluoromethyl group.

3. A catalyst composition comprising:
(A) the heteroatomic ligand compound of claim 1;
(B) a transition metal compound; and
(C) an organoaluminum compound.

4. The composition of claim 3, wherein the catalyst composition further comprises a hydrocarbon diluent or solvent.

5. The composition of claim 3, wherein the transition metal compound has the formula M(X¹)ₚ, wherein:
M is Fe, Co, or Cr;
p is an oxidation state of M; and
each X¹ independently is a monoanionic ligand.

6. The composition of claim 3, wherein the organoaluminum compound comprises an aluminoxane, an alkylaluminum compound, or any combination thereof.

7. The composition of claim 3, wherein a molar ratio of Al:ligand compound in the catalyst composition is in a range from 100:1 to 1,000:1.

8. An oligomerization process comprising:
(i) contacting ethylene, the catalyst composition of claim 3, an organic reaction medium, and optionally hydrogen, in an oligomerization reactor;
(ii) forming an oligomer product in the oligomerization reactor, the oligomer product comprising hexenes and octenes; and
(iii) discharging an effluent stream from the oligomerization reactor, the effluent stream comprising unreacted ethylene and the oligomer product.

9. The process of claim 8, wherein hydrogen is contacted in the oligomerization reactor.

10. The process of claim 8, wherein the catalyst composition is formed and then introduced into the oligomerization reactor.

11. The process of claim 8, wherein the catalyst composition is formed within the oligomerization reactor.

12. The process of claim 8, wherein hydrogen and ethylene are combined and introduced into the reactor separately from the catalyst composition or catalyst system components.

13. The process of claim 8, wherein the organic reaction medium comprises a saturated aliphatic hydrocarbon, an aromatic hydrocarbon, or any combination thereof.

14. The process of claim 8, wherein the organic reaction medium comprises cyclohexane.

15. A heteroatomic ligand compound having the following formula: 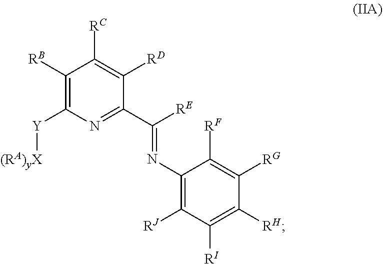
wherein:
X is P or S;
y is equal to 1 when X is S, and y is equal to 2 when X is P;
Y is O, NH, or CH₂;
R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and
each R^{A} independently is a C₁-C₁₈ hydrocarbyl group or a C₁-C₁₈ halogenated hydrocarbyl group.

16. A catalyst composition comprising:
(A) the heteroatomic ligand compound of claim 15;
(B) a transition metal compound; and
(C) an organoaluminum compound.

17. An oligomerization process comprising:
(i) contacting ethylene, the catalyst composition of claim 16, an organic reaction medium, and optionally hydrogen, in an oligomerization reactor;
(ii) forming an oligomer product in the oligomerization reactor, the oligomer product comprising hexenes and octenes; and
(iii) discharging an effluent stream from the oligomerization reactor, the effluent stream comprising unreacted ethylene and the oligomer product.

18. The process of claim 17, wherein the catalyst composition is formed within the oligomerization reactor.

19. A heteroatomic ligand transition metal compound complex having the following formula: 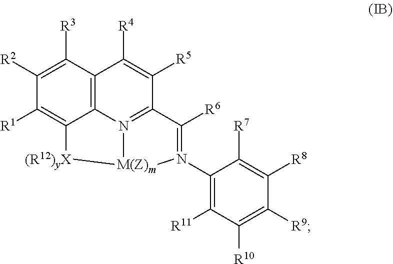
wherein:
X is P or S;
y is equal to 1 when X is S, and y is equal to 2 when X is P;
M is Fe, Co, or Cr;
m is an oxidation state of M;
each Z independently is H, a halogen, a C₁-C₁₈ hydrocarbyl group, or a C₁-C₁₈ halogenated hydrocarbyl group;
R¹ to R¹¹ independently are H, a halogen, a nitro group, a C₁-C₁₈ hydrocarbyl group, or a C₁-C₁₈ halogenated hydrocarbyl group, and R⁵ and R⁶ can be joined to form a ring or ring system; and
each R¹² independently is a C₁-C₁₈ hydrocarbyl group or a C₁-C₁₈ halogenated hydrocarbyl group.

20. A heteroatomic ligand transition metal compound complex having the following formula: 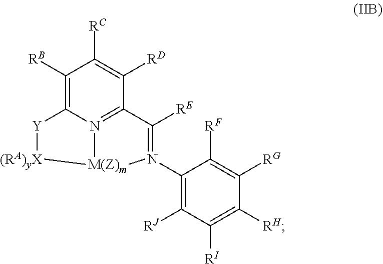
wherein:
X is P or S;
y is equal to 1 when X is S, and y is equal to 2 when X is P;
Y is O, NH, or CH₂;
M is Fe, Co, or Cr;
m is an oxidation state of M;
each Z independently is H, a halogen, a C₁-C₁₈ hydrocarbyl group, or a C₁-C₁₈ halogenated hydrocarbyl group;
R^{B} to R^{J} independently are H, a halogen, a nitro group, a C₁-C₁₈ hydrocarbyl group, or a C₁-C₁₈ halogenated hydrocarbyl group, and R^{D} and R^{E} can be joined to form a ring or ring system; and
each R^{A} independently is a C₁-C₁₈ hydrocarbyl group or a C₁-C₁₈ halogenated hydrocarbyl group.
